(19) United States
(12) Patent Application Publication
(10) Pub. No.: US 2024/0301504 A1
(43) Pub. Date: Sep. 12, 2024

(54) MULTIANALYTE BIOMARKERS FOR LUNG CANCER

(71) Applicant: The Curators of the University of Missouri, Columbia, MO (US)

(72) Inventor: Yves Chiswili Chabu, Columbia, MO (US)

(21) Appl. No.: 18/573,402

(22) PCT Filed: Jun. 22, 2022
(86) PCT No.: PCT/US2022/073094

§ 371 Date: Dec. 21, 2023

Related U.S. Application Data
(60) Provisional application No. 63/202,722, filed on Jun. 22, 2021.

(51) Int. Cl. C12Q 1/6886

(52) U.S. Cl. CPC C12Q 1/6886; C12Q 2600/158; C12Q 2600/178

(57) ABSTRACT

The present disclosure relates to methods of diagnosing and treating lung cancer using blood-borne analytes, the levels of which relate to the presence, stage and/or drug-resistant status of lung cancer in an individual. Such analytes are useful for indicating the presence, stage and/or drug-resistant status of lung cancer in an individual and to make treatment decisions for lung cancer patients. The present disclosure also discloses methods of treating lung cancer, some aspects of which include sensitizing lung cancer cells to treatment and decreasing resistance of lung cancer cells to treatment. Also disclosed are kits for practicing methods of the disclosure.

### CROSS REFERENCE TO RELATED APPLICATIONS

This application claims priority to U.S. Provisional Patent Application No. 63/202,722, filed Jun. 22, 2021, and PCT Application No. PCT/US2022/073094, filed on Jun. 22, 2022, which are hereby incorporated by reference in their entireties.

### STATEMENT REGARDING FEDERALLY SPONSORED RESEARCH & DEVELOPMENT

None.

### BACKGROUND OF THE INVENTION

Lung cancer causes the most cancer-dependent deaths worldwide, with 85% of lung cancer patients presenting with non-small cell lung cancer (NSCLC) and 70% of the cases diagnosed as late-stage disease. The 5-year survival rate for late-stage NSCLC is 2-5% compared to 92% for early-stage disease, underscoring the importance of early detection.

Current lung cancer screening involves the use of the Lung imaging reporting and data system (Lung-RADS). This classification system relies on chest low-dose computed tomography (LDCT) scans: based on the numbers, size, appearance, and location of detected lung nodules, Lung-RADS assigns a score where Lung-RADS 1 means no lung nodules, Lung-RADS 2 and 3 represent probably benign lung nodules, whereas suspicious nodules with highest risk of cancer are categorized as Lung-RADS4). However, LDCT has low specificity and a high false-positives rate (˜23%). For patients who undergo multiple rounds of LDCT screening, the cumulative false-positive rate is estimated at 38-50%. In addition to the unnecessary emotional distress, these over-diagnosed patients are subjected to costly and invasive follow ups before they are confirmed lung cancer free. There is a need for minimally invasive strategies that permit early and accurate detection of NSCLC.

Patients who are diagnosed with NSCLC are stratified to chemotherapy and/or immunotherapy, or targeted therapy based on the presence or absence of known NSCLC driver mutations in tissue biopsy analyses. Depending on patients' ethnicity, activating mutations of epidermal growth factor receptor (EGFR) tyrosine kinase is found in 15-50% of NSCLC. Patients harboring drug sensitizing EGFR mutations are treated with tyrosine kinase inhibitors or TKI. Some patients initially respond to tyrosine kinase inhibitors (TKI) but ultimately develop resistance: cancers acquire TKI-desensitizing EGFR mutations or activate compensatory signals, including WNT/β-catenin, the mechanistic target of rapamycin mTOR, and AKT signaling to drive cancer recurrence. Screening for genetic alterations that activate these signaling pathways provides a rationale for prioritizing treatment options that maximize the probability of achieving durable outcomes. However, it is becoming increasingly evident that cancer cells develop drug resistance via complex non-mutational mechanisms that involve emergent cell-cell interactions mediated by microRNAs (miRNAs). miRNAs are short (19-23) non-coding nucleotides that degrade protein transcripts and fundamentally impact signaling events. These miRNAs are released directly into the blood circulation (circulating miRNA) or secreted as extracellular vesicles (EV) cargo to elicit signaling events in target cells. The potential for EV miRNA as minimally invasive liquid biomarkers is now widely recognized.

### SUMMARY

One aspect is a method comprising, comparing the level of a lung cancer-dependent analyte in a test individual to the level of the lung cancer-dependent analyte in a control individual, and using the relative level of the lung cancer-dependent analyte in the test individual to: i) determine if the individual has lung cancer; ii) determine the stage of the individual's lung cancer; iii) determine if the individual's lung cancer is drug-resistant; and/or iv) treat the individual for lung cancer, wherein the cancer-dependent analyte is selected from the group consisting of CD9, CD63, has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p. In some aspects, the method may comprise determining the level of the lung cancer-dependent analyte in the test individual; determining the level of the lung cancer-dependent analyte in the control individual; and comparing the level of the lung cancer-dependent analyte in the test individual to the level of the lung cancer-dependent analyte in the control individual to determine the relative level of the lung cancer-dependent analyte in the test individual. In some aspects, the cancer-dependent analyte may be CD63 or CD9. In some aspects, the method may comprise comparing the level of the lung cancer-dependent analyte in the test individual with the level of a cancer-independent analyte in the test individual, there producing a test ratio, and using the test ratio to determine if the individual has lung cancer and/or treat the individual for lung cancer. The cancer-dependent analyte may be CD63 and the test individual may be identified as having lung cancer, or a as having stage IVlung cancer, when the CD63/cancer-independent analyte test ratio is at least 1.0, at least 1.5, at least 2.0 at least 3.0, or at least 4.0. The cancer-dependent analyte may be CD9 and the test individual is identified as having lung cancer, or as having stage IV lung cancer, when the CD9/cancer-independent analyte test ratio in the test individual is at least 1.5, at least 2.0, at least 2.5, or at least 3.0. In some aspects, the method may comprise comparing the test ratio with the ratio of the cancer-dependent analyte to the cancer-independent analyte in the control individual (aka control ratio), and using the comparison to: i) determine if the individual has lung cancer; ii) determine the stage of the individual's lung cancer; iii) determine if the individual's lung cancer is drug-resistant; and/or iv) treat the individual for lung cancer. In these aspects, the cancer-dependent analyte may be CD63, and the test individual is identified as having lung cancer, or as having stage IV lung cancer, when the ratio of the CD63/cancer-independent analyte test ratio to the CD63/cancer-independent analyte control ratio is at least 2.0, at least 2.5, at least 3.0, at least 3.5, at least 4.0, at last 4.5 at least 5.0, or at least 6.0. The cancer-dependent analyte may be CD9, and the test individual is identified as having lung cancer, or as having stage IV, when the ratio of the CD9/cancer-independent analyte test ratio to the CD9/cancer-independent analyte control ratio is at least 1.5 or at least 2.0. In some aspects, the method may comprise comparing the level of a first cancer-dependent analyte in the test individual, with the level of a second cancer-dependent analyte in the test individual, there by obtaining a test ratio; and, using the test ratio to identify the individual as having lung cancer. The first cancer-dependent analyte may be CD9 and the second cancer-dependent analyte may be CD63, and the test individual is identified as having lung cancer, or as having stage IV lung cancer, when the CD9/CD63 test ratio in the test individual is less than 2.0, 1.5, 1.0, or 0.5. The method may comprise comparing the test ratio from the individual with a control ratio produced using the levels of the same analytes used to produce the test ratio, but from a control individual. Thus, the cancer-dependent analyte/cancer-independent analyte test ratio may be compared with a cancer-dependent analyte/cancer-independent analyte control ratio, and the comparison used to identify the individual as having lung cancer. The first cancer-dependent analyte may be CD9, and the second cancer-dependent analyte may be CD63, and the test individual is identified as having lung cancer when the ratio of the test ratio to the control ratio is at least 4.0, at least 5.0, at least 6.0, at least 7.0, or at least 8.0. The first cancer-dependent analyte may be EV CD9, and the second cancer-dependent analyte may be EV CD63, and the test individual is identified as having lung cancer, or as having lung cancer, when the ratio of the test ratio to the control ratio is at least 4.0, at least 5.0, at least 6.0, at least 7.0, or at least 8.0.

In these aspects, the cancer-independent analyte may be flotillin, Tsg101, or Hsp70. In these aspects, the cancer-dependent analyte and/or the cancer-independent analyst may, independently, be a circulating analyte, an EV-associated analyte, or may comprise circulating analyte and EV-associated analyte. In some aspects, the control individual is the same as the test individual, but the control ratio is determined at a time when the individual was known to be free of lung cancer.

One aspect of the disclosure is a method of identifying a test individual as having lung cancer, comprising comparing the level of a cancer-dependent analyte in the test individual, with the level of the cancer-dependent analyte in a control individual, and identifying the individual as having lung cancer if the level of the cancer-dependent analyte in the test individual is significantly different than the level of the cancer-dependent analyte in the control individual, wherein the cancer-dependent analyte comprises an miRNA selected from the group consisting of has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p. The miRNA may comprise a circulating miRNA or an EV-associated miRNA.

The cancer-dependent analyte may comprise an EV-associated miRNA selected from the group consisting of has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, and has-miR-155-5p, wherein the individual is identified as having lung cancer if the level of the EV-associated miRNA in the test individual is significantly greater than the level of the EV-associated miRNA in the control individual. The cancer-dependent analyte may comprise an EV-associated miRNA selected from the group consisting of has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, and has-miR-29c-3p, wherein the individual is identified as having lung cancer if the level of the EV-associated miRNA in the test individual is significantly less than the level of the EV-associated miRNA in the control individual. The cancer-dependent analyte may comprise a circulating miRNA selected from the group consisting of has-miR-423-5p, has-miR-486-5p, has-miR-320a-3p, has-miR-185-5p, has-miR-99a-5p, has-Let-7b-5p, has-miR-22-3p, has-miR-122-5p, has-miR-192-5p, has-miR-148a-3p, has-miR-21-5p, and has-miR-143-3p, wherein the individual is identified as having lung cancer if the level of the circulating miRNA in the test individual is significantly greater than the level of the circulating miRNA in the control individual. The cancer-dependent analyte may comprise a circulating miRNA selected from the group consisting of has-miR-184, has-miR-3976, has-miR-4259, has-miR-598-3p, has-miR-7-5p, has-miR-223-3p, has-miR-16-5p, has-miR-22-3p, has-miR-425-5p, has-miR-146a-5p, has-miR-92b-3p, has-miR-423-5p, has-miR-320a-3p, has-miR-486-5p, has-let-7i-5p, has-miR-451a, has-miR-142-5p, and has-let-7c-5p, wherein the individual is identified as having lung cancer if the level of the circulating miRNA in the test individual is significantly less than the level of the circulating miRNA in the control individual. The cancer-dependent analyte may comprise let-7b-5p miRNA, and the individual is identified as having lung cancer, or as having stage IV lung cancer, if the level of let-7b-5p miRNA in the test individual is at least 2-fold, at least 3-fold, at least 4-fold, or at least 5-fold higher, or at least 0.5 logs or at least 1.0 log higher, than the level of let-7b-5p miRNA in the control individual. The cancer-dependent analyte may comprise let-7b-5p miRNA, and the individual is identified as having lung cancer, or as having stage IV lung cancer, if the level of let-7b-5p miRNA in the test individual is at least 20%, at least 30%, at least 40%, at least 50%, at least 60%, at least 70%, at least 80% or at least 90%, greater, or at least 0.5 logs or at least 1.0 log higher, than the level of let-7b-5p miRNA in the control individual. The cancer-dependent analyte may comprise miR-184 miRNA, or miR-22-3p miRNA, and the individual is identified as having lung cancer, as having stage IV lung cancer, and/or as having a drug resistant cancer, if the level of miR-184 miRNA, or miR-22-3p miRNA, in the test individual is at least 20%, at least 30%, at least 40%, at least 50%, at least 60%, at least 70%, at least 80% or at least 90%, or at least 0.5 logs or at least 1.0 log, lower than the level of miR-184 miRNA in the control individual. In these aspects, the miRNA may comprise a circulating miRNA or an EV-associated miRNA.

One aspect is a method of decreasing the treatment-resistance of a lung cancer in a cancer patient, comprising modulating the expression of let-7b-5p miRNA, miR-184 miRNA, and/or miR-22-3p miRNA in the cancer patient. The method may comprise decreasing the level of let-7b-5p miRNA in the cancer patient, and/or increasing the level of miR-184 miRNA and/or miR-22-3p miRNA in the cancer patient, which may comprise administering to the cancer patient miR-184 miRNA and/or miR-22-3p miRNA, or a vector encoding miR-184 miRNA and/or miR-22-3p miRNA.

One aspect is a method of sensitizing lung cancer cells to treatment in a cancer patient, comprising modulating the expression of let-7b-5p miRNA, miR-184 miRNA, and/or miR-22-3p miRNA in the cancer patient. The method may comprise decreasing the level of let-7b-5p miRNA in the cancer patient, and/or increasing the level of miR-184 miRNA and/or miR-22-3p miRNA in the cancer patient, which may comprise administering to the cancer patient miR-184 miRNA and/or miR-22-3p miRNA, or a vector encoding miR-184 miRNA and/or miR-22-3p miRNA.

One aspect is a method of treating an individual for lung cancer, comprising comparing the level of miR-184 miRNA in the test individual with the level miR-184 miRNA in the control individual; and/or comparing the level of miR-22-3p miRNA in the test individual with the level miR-22-3p miRNA in the control individual; wherein if the level of miR-184 miRNA in the test individual is significantly less than the level of miR-184 miRNA in the control individual; and/or if the level of miR-22-3p miRNA in the test individual is significantly less than the level of miR-22-3p miRNA in the control individual; administering to the individual a tyrosine kinase inhibitor and an inhibitor of one or more proteins in the p13k/Akt pathway. The tyrosine kinase inhibitor may be selected from the group consisting of Erlotinib, Afatinib, Gefiiniband, Osimertinib, and combinations thereof.

One aspect of the disclosure is a kit comprising: i) reagents for use in determining the level of at least one cancer-dependent analyte; and, ii) instructions for using the reagents to determine the level of the at least at least one cancer-dependent analyte; wherein the cancer-dependent analyte is selected from the group consisting of CD9, CD63, has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p.

### BRIEF DESCRIPTION OF THE DRAWINGS

FIGS. 1A-1C show that CD9 and CD63, but not CD81, are enriched on NSCLC EV. FIG. 1A shows a representative transmission electron micrograph of the isolated extracellular vesicles (EV). FIGS. 1B & 1C show nanoparticle tracking analyses (NTA) data showing EV concentrations (particles/mL) (FIG. 1B) and size distribution (FIG. 1C). One-way ANOVA analysis was performed to determine statistical significance, *<0.05.

FIGS. 2A & 2B show Western blot analysis of EV markers. FIG. 2A shows a Western blot image showing the abundance of EV markers (CD81, CD63, CD9, and Flotillin) in EV isolated from blood samples obtained from screening controls (LNG-RADS II) or LNG-RADS IV individuals. FIG. 2B shows a Western blot images from triplicate samples showing CD9 and CD63 protein levels in EV extracted from LNG RADS II or confirmed cancer patients (IV+) plasma or disease-free LNG-RADS IV (IV−) individuals. Flotillin protein levels are used as loading controls. These experiments are shown in triplicates.

FIGS. 3A-3C show relative quantification of EV biomarkers. FIGS. 3A & 3B show relative quantification of CD9 and CD63 protein levels, respectively, in the Western blot shown of FIG. 2B. Protein levels are denoted as ratio of pixel intensities mean values quantified from each of the three replicates and normalized to Flotillin. FIG. 3C shows the ratio of CD9:CD63 protein band intensities presented in FIG. 2B. Quantification of band intensities (pixels) was performed using ImageJ. Bars represent data as mean±SD. p values are derived from t-test. *p<0.05, **p<0.01, ***p<0.001.

FIGS. 4A-4L Identification of unique EV and circulating miRNA profiles in Lung-RADS4 cancer patients. Next Generation Sequencing (NGS) of blood EV or circulating miRNA identified Let-7b-5p, miR-184, and miR-22-3p as differentially expressed in NSCLC patients compared to controls. Let-7b-5p (FIGS. 4A & 4B) and mir-184 (FIGS. 4C & 4D) are deregulated in EV and mir-22-3p in plasma (FIGS. 4I & 4J). The relative abundance of the indicated miRNA in screening controls versus confirmed diseased patients only or versus all lung-RADS2 plus false positive lung-RADS4 are shown in FIGS. 4A-4D and FIGS. 4I & 4J, respectively. P values were Benjamini-Hochberg adjusted. *p<0.1, **p<0.05, ***p<0.01, ****p<0.001. Note that 2-3 patient samples with undetectably low reads were excluded from the analysis. Corresponding Reads per million (RPM) were used to plot miRNA levels in confirmed cancer patients versus disease-free individuals and to perform ROC analysis shown in FIGS. 4E-4H, FIGS. 4K, and FIG. 4L. EdgeR generalized linear models (GLM) were used to assess significance of miRNA regulations.

FIG. 5 shows multiple logistic regression analysis was performed to determine the combined classification performance of the three miRNA biomarker candidates. Statistical significance of miRNA regulations was determined by EdgeR GLM, as indicated above.

FIGS. 6A-6C show quantitative Polymerase Chain Reaction (qPCR) data showing expression fold changes (means) of let-7b-5p (FIG. 6A) or miRNA-184 (FIG. 6B) or miRNA-21-5p (NOT SHOWN) or miRNA-22-3p (FIG. 6C) in EV samples obtained from confirmed cancer patients (IV+) versus screening controls (II). RNA samples were pooled from 14 cancer patients and 14 randomly selected screening individuals. Expression was normalized to GAPDH. Error bars denote SD values. P values are derived from student t-test analyses.

FIG. 7 shows a Venn diagram showing the number unique of shared protein targets between the selected miRNAs.

FIG. 8 shows a MIRNET star-network showing proteins that are targeted by at least two of the three miRNAs (blue squares).

FIG. 9 shows a KEGG classification analyses of the identified proteins showing a convergence onto PI3K-AKT-mTOR and WNT/B-catenin signaling pathways.

FIG. 10 shows a Reactome classification analyses of the identified proteins.

FIG. 11A-11D show analysis of A549 cells treated with EV. FIG. 11A shows Western blot imaging from A549 cells treated with equal quantity of EV from cancer patients or from high-risk screening controls. Blots were stained against β-catenin to detect WNT signaling levels or GAPDH as a loading control. FIGS. 11B & 11C show Western blot images from A549 cells cultured in standard media or media conditioned with PBMC in the absence or presence of patients EV (FIG. 11B, lanes 1-4). Also, A549 cells were treated directly with cancer patients or control EV (FIG. 11B, lanes 5 and 6). Blots were stained against phospho-AKT1 (FIG. 11B) or phospho-mTOR (FIG. 11C) or GAPDH as a loading control (FIGS. 11B & 11C). A549 cell numbers from supernatant transfer experiments (FIG. 11B, lanes 1-4) are shown in “h” as average cell numbers from triplicate experiments. Error bars denote SD values. P values are derived from student t-test analyses.

FIGS. 12A & 12B show qPCR data showing mean expression fold changes of miR-184 (FIG. 12A) or miR-22-3p (FIG. 12B) in H1975 cells transfected with miR-184 (FIG. 12A) or miR-22-3p inhibitors (FIG. 12A) compared to untreated H1975 control cells. Expression was normalized to GAPDH.

FIGS. 13A-13C show the effect of treating cells with miRNA inhibitors. FIG. 13A shows Western blot images from H1975 cells left untreated or treated with miR-22-3p or miR-184 inhibitors and blotted against phospho-AKT1 or GAPDH (loading control). FIG. 13B shows an image of a Western blot from untreated H1975 cells or H1975 cells treated with equal portions of EV from screening controls (RADS-II) or with inhibitors against miR-22-3p and/or miR-184 inhibitors followed with Osimertinib (100 nM) treatments. Western blots were stained against phospho-AKT or GAPDH (loading control). FIG. 13C shows a graph illustrating the proportion of H1975 cell death across the indicated conditions using Trypan blue exclusion assays. These cell death assays were performed in triplicates and the results are shown as the average proportion (percentage) of dead cell across replicates for each treatment conditions. Error bars denote standard deviations and p values were derived from t-tests.

FIGS. 14A-14C show survivorship comparison data using miR-184 and/or miR-22-3p expression data in TCGA-LUAD and the Bioconductor tool TCGA Biolinks RTCGA R packages. The “surv_cutpoint” function of the “survminer” R package was used to identify high versus low expressing patients' samples for miR-22-3p (FIG. 14A) or miR-184 (FIG. 14B) or both (FIG. 14C) in Cox regression analyses. Survminer uses selected rank statistics to determine the optimal cut-point of a continuous variable in an unbiased manner.

FIGS. 15A & B show proposed model summarizing the role of mir-184/mir-22 in Osimertinib drug response. EV (mir-184) and circulating (mir-22) plasma miRNAs cooperatively target and modulate AKT activity. (FIG. 15A) Cancer-free high-risk individuals up-regulate EV mir-184 and circulating mir-22, keeping AKT levels generally low. In the context of genetic driver mutations, this low AKT activity delays oncogenesis. Similarly, cancer patients with high EV mir-184 and circulating mir-22-3p maintain AKT below an activity threshold required for AKT-mediated drug resistance, leading to positive drug response. However, AKT baseline activity is elevated in patients with low mir-184/mir-22 levels such that even a modest stimulation of AKT drives AKT above the drug-resistance activity threshold, leading to relapses and poor clinical response (FIG. 15B).

### DETAILED DESCRIPTION OF THE INVENTION

The present disclosure relates to methods of diagnosing and treating lung cancer. More specifically, the present disclosure provides blood-borne analytes, the levels of which relate to the presence and/or the stage or drug-resistant status of lung cancer in an individual. Thus, these analytes may be used to indicate the presence, stage and/or drug-resistant status of lung cancer in an individual and to make treatment decisions for lung cancer patients. Accordingly, methods of the disclosure may generally be practiced by determining and/or comparing the relative levels of one or more analytes disclosed herein, and using such relative levels to: identify an individual as having lung cancer; determine the stage of a lung cancer; determine the drug-resistant status of a lung cancer; and/or treat an individual for lung cancer. Examples of analytes useful for practicing the disclosed methods include, but are not limited to, CD9, CD63, flotillin, Tsg101, Hsp70, has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed. All publications mentioned herein are incorporated herein by reference to disclose and describe the methods and/or materials in connection with which the publications are cited.

Unless defined otherwise, all technical and scientific terms used herein have the same meaning as commonly understood by one of ordinary skill in the art to which the invention belongs. Although any methods and materials similar or equivalent to those described herein can also be used in the practice or testing of the present invention, the preferred methods and materials are now described. Terms and phrases, which are common to the various aspects disclosed herein, are defined below.

One aspect of the disclosure is a method comprising, comparing the level of a lung cancer-dependent analyte in a test individual to the level of the lung cancer-dependent analyte in a control individual, and using the relative level of the lung cancer-dependent analyte in the test individual to: i) determine if the individual has lung cancer; ii) determine the stage of the individual's lung cancer; iii) determine if the individual's lung cancer is drug-resistant; and/or iv) treat the individual for lung cancer. In some aspects, the method may comprise determining the level of the lung cancer-dependent analyte in the test individual; determining the level of the lung cancer-dependent analyte in the control individual; and comparing the level of the lung cancer-dependent analyte in the test individual to the level of the lung cancer-dependent analyte in the control individual to determine the relative level of the lung cancer-dependent analyte in the test individual. In some aspects, the method may comprise comparing the level of the lung cancer-dependent analyte in the test individual with the level of a cancer-independent analyte in the test individual, thereby obtaining the ratio of the cancer-dependent analyte to the cancer-independent analyte in the test individual (aka test ratio), and using the test ratio to: i) determine if the individual has lung cancer; ii) determine the stage of the individual's lung cancer; iii) determine if the individual's lung cancer is drug-resistant; and/or iv) treat the individual for lung cancer. In some aspects, the method may comprise comparing the test ratio with the ratio of the cancer-dependent analyte to the cancer-independent analyte in the control individual (aka control ratio), and using the comparison to: i) determine if the individual has lung cancer; ii) determine the stage of the individual's lung cancer; iii) determine if the individual's lung cancer is drug-resistant; and/or iv) treat the individual for lung cancer. In some aspects, the cancer-dependent analyte may be selected from the group consisting of CD9, CD63, has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p. In some aspects, the cancer-independent analyte may be flotillin. In some aspects, the cancer-dependent analyte may be a circulating analyte. In some, the cancer-dependent analyte may be an EV-associated analyte. In some aspects, the cancer-dependent analyte may comprise circulating analyte and EV-associated analyte. In some aspects, the cancer-independent analyte may be circulating analyte. In some aspects, the cancer-independent analyte may be EV-associated analyte. In some aspects, the cancer-independent analyte may comprise circulating analyte and EV-associated analyte.

“Analyte” means any substance within a biological sample that can be detected and/or measured. An analyte may be a chemical or a biological substance, such as a protein, a nucleic acid molecule (e.g., RNA), a lipid, a lipoprotein, or a sugar. A “cancer-dependent analyte” is a chemical or a biological substance in an individual, the level of which is altered (e.g., increased, decreased) when cancer is present in the individual, when a particular stage of cancer is present in the individual, and/or when a drug-resistant cancer is present in the individual. Examples of cancer-dependent analytes useful for practicing methods of the disclosure include, but are not limited to, CD9 protein (“CD9”), CD63 protein (“CD63”), has-let-7b-5p, has-miR-26a-5p, has-mi R-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p. A “cancer-independent analyte” is a chemical or a biological substance within an individual, the level of which is independent of the presence, stage and/or status of a cancer. Examples of cancer-independent analytes useful for practicing methods of the disclosure include, but are not limited to, flotillin, Tsg101, and Hsp70.

The term “level” generally refers to the amount of an analyte and may be indicated using any appropriate quantifier. For example, “level” may refer to the concentration of analyte (e.g., ng/ml, nM, etc.), or it may refer to the relative amount of an analyte (i.e., the amount relative to a known amount of a reference molecule). Any method of detection and/or measurement appropriate for a given molecule may be used in methods of the disclosure to determine the level of an analyte. For example, the level of a protein may be determined using an assay such as an ELISA, while the level of miRNA may be determined using an assay such as quantitative reverse transcription polymerase chain reaction (qRT-PCR). It will be understood that, in some instances, due to sample to sample variation, the level of an analyte may be normalized to a “housekeeping” protein, such as glyceraldehyde-3-phosphate dehydrogenase (GAPDH). Levels of analytes may be determined in a sample from an individual, such as a blood sample, a serum sample, a plasma sample, a tissue sample, or a saliva sample.

The terms “individual”, “subject”, and “patient” are well-recognized in the art and are herein used interchangeably to refer to any animal susceptible to developing lung cancer. Examples of an individual include, but are not limited to, humans and other primates, including non-human primates such as chimpanzees and other apes and monkey species; farm animals such as cattle, sheep, pigs, goats and horses; domestic mammals such as dogs and cats; and laboratory animals, including rodents such as mice, rats and guinea pigs. The terms individual, subject, and patient by themselves, do not denote a particular age, sex, race, and the like. Thus, individuals of any age, whether male or female, are intended to be covered by the present disclosure and include, but are not limited to the elderly, adults, children, babies, infants, and toddlers.

A “test individual” means an individual for whom it is desired to determine their cancer status, to determine the stage of a cancer in the individual, to determine if the individual's cancer is drug-resistant, and/or to treat for cancer. “Cancer status” refers to the presence or absence of cancer, or to characteristics of a cancer. For example, cancer status may refer to the presence or absence of cancer, the presence of absence of a particular type or stage (e.g., stage IV″) of cancer, or to whether not a cancer is drug-resistant. “Drug resistant” means a cancer has become tolerant to a pharmaceutical treatment (e.g., chemotherapy). Thus, a drug resistant cancer cell can survive (not be killed by) and possibly replicate in the presence of the pharmaceutical treatment to which it is resistant. “Drug resistant” encompasses all means by which a cancer cell my become tolerant to a pharmaceutical treatment, such as drug inactivation, drug target alteration, drug efflux, and cell death inhibition. A “control individual” is an individual known to be free of cancer or known to be free of a particular stage or type of cancer (e.g., stage IV cancer or drug resistant cancer). In certain methods of the disclosure, the level of an analyte in a test individual may be compared to the level of the test analyte in a control individual. It should be understood that the level of an analyte, or the ratio between analyte levels, in a control individual need not be determined at the same time, or at all, in order to practice methods of the disclosure. For example, the level of an analyte, or the ratio between analyte levels, in a control individual may be historical. Thus, the level of an analyte, or the ratio between analyte levels, in a control individual may be obtained from a chart, a table, or the like.

As used herein the term “cancer” refers to a physiological condition in mammals that is typically characterized by unregulated cell growth. Lung cancer means any cancer that originates in tissue of the lungs or other organs and spreads to lung tissues. Lung cancers are often divided into the two main categories of small-cell lung cancer (SCLC), also called oat cell cancer, and non-small-cell lung cancer (NSCLC). NSCLC may be further divided into three major types, squamous cell carcinoma (SCC), adenocarcinoma and large cell carcinomas. NSCLCs may further be staged, which is a clinical process that is generally based on the size of the main tumor and the presence of metastasis and the distance of their spread. Staging of a cancer may be very detailed, although in general, a stage I or stage II cancer is considered “early stage”, while a stage III or stage IV cancer is considered “late stage”. Methods of NSCLC staging are well known in the art. In one aspect, the lung cancer may be non-small cell lung cancer (NSCLC). In one aspect, the lung cancer may be lung adenocarcinoma (AC). In one aspect, the lung cancer may be lung squamous cell carcinoma (SCC). In one aspect, the lung cancer may be an “early stage” (I or II) NSCLC. In one aspect, the lung cancer may be a “late stage” (III or IV) NSCLC. In one aspect, the lung cancer may be a mixture of early and late stages and types of NSCLC.

“Extracellular vesicle” (EV) refers to a nano to micro-sized membraned vesicle released by essentially all types of cells. EVs comprise a lipid bilayer, range in size from 30 to 10,000 nm in diameter, and may comprise various biomolecules, such as proteins, nucleic acid molecules (e.g., RNA such as miRNA) and lipids, from the cell from which the EV originated. Thus, EVs may comprise bioactive content that can influence the tumor microenvironment as well as the microenvironment at tumor-distal sites. In some aspects, EVs of the disclosure may be at least 70 nm in size, or at least 100 nm or at least 110 nm in size. In some aspects, EVs of the disclosure may be in the range of 70 nm to 100, 70-150 nm, or 70 nm to 1000 nm in size.

An “EV-associated analyte” means an analyte that is found to be present in a sample of EV after the EV has been isolated from a biological sample, such as blood or tissue, using a purification process. As used herein, the term “isolated” refers to a biological material, such as an EV, that has been removed from its original naturally occurring environment. An “isolated EV” of the disclosure has, at least, been removed from a sample of blood taken from an individual. Such removal may comprise filtration using a filter (e.g., paper) that retains the EVs, centrifugation, and/or chromatography, including affinity chromatography (AC) and size exclusion chromatography (SEC). In some aspects of the disclosure, an isolated EV of the disclosure may be obtained by centrifugation of a blood sample, followed by purification comprising SEC, which may comprise using media yielding EVs at least 70 nm in size, or at least 100 nm or 110 nm in size. Such a method would produce a sample of EVs that exclude EVs smaller than about 110 nm, about 100 nm, or about 70 nm in size. In some aspects, SEC may yield EVs in the range of 70 nm to 100, 70-150 nm, or 70 nm to 1000 nm, in size.

One aspect of the disclosure is a method of identifying a test individual as having lung cancer, comprising comparing the level of a cancer-dependent analyte selected from the group consisting of CD63 and CD9 in the test individual, with the level of the cancer-dependent analyte in a control individual, wherein if the level of the cancer-dependent analyte in the test individual is significantly greater than the level of the cancer-dependent analyte in the control individual, identifying the individual as having lung cancer. In some aspects, the method may comprise comparing the level of CD63 or CD9 in the test individual with the level of a cancer-independent analyte in the test individual to produce a test ratio (e.g., a CD63/cancer-independent analyte test ratio or a CD9/cancer-independent analyte test ratio), and using the test ratio to identify the individual as having lung cancer. In some aspects, the cancer-dependent analyte may be CD63 and the test individual is identified as having lung cancer when the CD63/cancer-independent analyte test ratio is at least 1.0, at least 1.5, at least 2.0 at least 3.0, or at least 4.0. In some aspects, the cancer-dependent analyte may be CD9 and the test individual is identified as having lung cancer when the CD9/cancer-independent analyte test ratio in the test individual is at least 1.5, at least 2.0, at least 2.5, or at least 3.0. In some aspects, the cancer-independent analyte may be flotillin. Thus, in some aspects, the test individual is identified as having lung cancer when the CD63/flotillin test ratio in the test individual is greater than at least 1.0, at least 1.5, at least 2.0 at least 3.0, or at least 4.0. In some aspects, the test individual is identified as having lung cancer when the CD9/flotillin test ratio in the test individual is at least 1.5, at least 2.0, at least 2.5, or at least 3.0.

In some aspects, the method may comprise comparing the test ratio from the individual with a control ratio produced using the levels of the same analytes used to produce the test ratio, but from a control individual. Thus, the CD63/cancer-independent analyte test ratio may be compared with a CD63/cancer-independent analyte control ratio, and the comparison used to identify the individual as having lung cancer. Similarly, the CD9/cancer-independent analyte test ratio may be compared with a CD9/cancer-independent analyte control ratio, and the comparison used to identify the individual as having lung cancer. In some aspects, the individual is identified as having lung cancer if the test ratio is significantly larger than the control ratio. In some aspects, the cancer-dependent analyte may be CD63, and the test individual is identified as having lung cancer when the ratio of the CD63/cancer-independent analyte test ratio to the CD63/cancer-independent analyte control ratio is at least 2.0, at least 2.5, at least 3.0, at least 3.5, at least 4.0, at last 4.5 at least 5.0, or at least 6.0. In some aspects, the cancer-dependent analyte may be CD9, and the test individual is identified as having lung cancer when the ratio of the CD9/cancer-independent analyte test ratio to the CD9/cancer-independent analyte control ratio is at least 1.5 or at least 2.0. In some aspects, the cancer-independent analyte may be flotillin, and the test individual may be identified as having lung cancer if the test ratio is significantly larger than the control ratio. In some aspects, the cancer-dependent analyte may be CD63, the cancer-independent analyte may be flotillin, and the test individual is identified as having lung cancer when the ratio of the CD63/flotillin test ratio to the CD63/flotillin control ratio is at least 2.0, at least 2.5, at least 3.0, at least 3.5, at least 4.0, at last 4.5 at least 5.0, or at least 6.0. In some aspects, the cancer-dependent analyte may be CD9, the cancer-independent analyte may be flotillin, and the test individual is identified as having lung cancer when the ratio of the CD63/flotillin test ratio to the CD63/flotillin control ratio is at least 1.5 or at least 2.0. In some aspects, the CD63 or CD9 may be circulating CD63 or circulating CD9. In some aspects, the CD63 or CD9 may be EV-associated CD63 or EV-associated CD9. In some aspects, the CD63 may comprise circulating CD63 and EV-associated CD63. In some aspects, the CD9 may comprise circulating CD9 and EV-associated CD9. In some aspects, the cancer-independent analyte may be circulating cancer-independent analyte. In some aspects, the cancer-independent analyte may be EV-associated cancer-independent analyte. In some aspects, the cancer-independent analyte may comprise circulating cancer-independent analyte and EV-associated cancer-independent analyte. In some aspects, the control individual is the same as the test individual, but the control ratio is determined at a time when the individual was known to be free of lung cancer.

One aspect of the disclosure is a method of identifying a test individual as having lung cancer, comprising comparing the level of a first cancer-dependent analyte in the test individual, with the level of a second cancer-dependent analyte in the test individual, there by obtaining a test ratio; and, using the test ratio to identify the individual as having lung cancer. In some aspects, the first cancer-dependent analyte may be CD9 and the second cancer-dependent analyte may be CD63, and the test individual is identified as having lung cancer when the CD9/CD63 test ratio in the test individual is less than 2.0. In some aspects, the first cancer-dependent analyte may be CD9, and the second cancer-dependent analyte may be CD63, and the test individual is identified as having lung cancer when the CD9/CD63 test ratio in the test individual is less than 1.5. In some aspects, the first cancer-dependent analyte may be CD9, and the second cancer-dependent analyte may be CD63, and the test individual is identified as having lung cancer when the CD9/CD63 test ratio in the test individual is less than 1.0. In some aspects, the first cancer-dependent analyte may be CD9, and the second cancer-dependent analyte may be CD63, and the test individual is identified as having lung cancer when the CD9/CD63 test ratio in the test individual is less than 0.5. In some aspects, the first cancer-dependent analyte and/or the second cancer-dependent analyte may be circulating analytes. In some aspects, the first cancer-dependent analyte and/or the second cancer-dependent analyte may be EV-associated analytes. In some aspects, the first cancer-dependent analyte and/or the second cancer-dependent analyte may comprise circulating analyte and EV-associated analyte. In some aspects, the first cancer-dependent analyte may be EV-associated CD9 (EV CD9), and the second cancer-dependent analyte may be EV-associated CD63 (EV CD63), and the test individual is identified as having lung cancer when the EV CD9/EV CD63 ratio in the test individual is less than 2.0, less than 1.5, less than 1.0, or less than 0.5.

In some aspects, the method may comprise comparing the test ratio from the individual with a control ratio produced using the levels of the same analytes used to produce the test ratio, but from a control individual. Thus, the cancer-dependent analyte/cancer-independent analyte test ratio may be compared with a cancer-dependent analyte/cancer-independent analyte control ratio, and the comparison used to identify the individual as having lung cancer. In some aspects, the individual is identified as having lung cancer if the test ratio is significantly larger than the control ratio. In some aspects, the individual is identified as having lung cancer if the control ratio is significantly smaller than the test ratio. In some aspects, the first cancer-dependent analyte may be CD9, and the second cancer-dependent analyte may be CD63, and the test individual is identified as having lung cancer when the ratio of the test ratio to the control ratio is at least 4.0, at least 5.0, at least 6.0, at least 7.0, or at least 8.0. In some aspects, the first cancer-dependent analyte may be EV CD9, and the second cancer-dependent analyte may be EV CD63, and the test individual is identified as having lung cancer when the ratio of the test ratio to the control ratio is at least 4.0, at least 5.0, at least 6.0, at least 7.0, or at least 8.0. In some aspects, the control individual is the same as the test individual, but the control ratio is determined at a time when the individual was known to be free of lung cancer.

One aspect of the disclosure is a method of identifying a test individual as having lung cancer, comprising comparing the level of a cancer-dependent analyte in the test individual, with the level of the cancer-dependent analyte in a control individual, and identifying the individual as having lung cancer if the level of the cancer-dependent analyte in the test individual is significantly different than the level of the cancer-dependent analyte in the control individual, wherein the cancer-dependent analyte comprises an miRNA selected from the group consisting of has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p. In some aspects, the miRNA comprises a circulating miRNA. In some aspects, the miRNA comprises an EV-associated miRNA. In some aspects, the miRNA comprises circulating miRNA and EV-associated miRNA. In some aspects, the control individual is the same as the test individual, but the control ratio is determined at a time when the individual was known to be free of lung cancer.

In some aspects, the cancer-dependent analyte comprises an EV-associated miRNA selected from the group consisting of has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, and has-miR-155-5p, wherein the individual is identified as having lung cancer if the level of the EV-associated miRNA in the test individual is significantly greater than the level of the EV-associated miRNA in the control individual.

In some aspects, the cancer-dependent analyte comprises an EV-associated miRNA selected from the group consisting of has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, and has-miR-29c-3p, wherein the individual is identified as having lung cancer if the level of the EV-associated miRNA in the test individual is significantly less than the level of the EV-associated miRNA in the control individual.

In some aspects, the cancer-dependent analyte comprises a circulating miRNA selected from the group consisting of has-miR-423-5p, has-miR-486-5p, has-miR-320a-3p, has-miR-185-5p, has-miR-99a-5p, has-Let-7b-5p, has-miR-22-3p, has-miR-122-5p, has-miR-192-5p, has-miR-148a-3p, has-miR-21-5p, and has-miR-143-3p, wherein the individual is identified as having lung cancer if the level of the circulating miRNA in the test individual is significantly greater than the level of the circulating miRNA in the control individual.

In some aspects, the cancer-dependent analyte comprises a circulating miRNA selected from the group consisting of has-miR-184, has-miR-3976, has-miR-4259, has-miR-598-3p, has-miR-7-5p, has-miR-223-3p, has-miR-16-5p, has-miR-22-3p, has-miR-425-5p, has-miR-146a-5p, has-miR-92b-3p, has-miR-423-5p, has-miR-320a-3p, has-miR-486-5p, has-let-7i-5p, has-miR-451a, has-miR-142-5p, and has-let-7c-5p, wherein the individual is identified as having lung cancer if the level of the circulating miRNA in the test individual is significantly less than the level of the circulating miRNA in the control individual.

In some aspects, the cancer-dependent analyte comprises let-7b-5p miRNA, and the individual is identified as having lung cancer if the level of let-7b-5p miRNA in the test individual is significantly greater than the level of let-7b-5p miRNA in the control individual. In some aspects, the cancer-dependent analyte comprises let-7b-5p miRNA, and the individual is identified as having lung cancer if the level of let-7b-5p miRNA in the test individual is at least 2-fold, at least 3-fold, at least 4-fold, or at least 5-fold higher than the level of let-7b-5p miRNA in the control individual. In some aspects, the cancer-dependent analyte comprises let-7b-5p miRNA, and the individual is identified as having lung cancer if the level of let-7b-5p miRNA in the test individual is at least 20%, at least 30%, at least 40%, at least 50%, at least 60%, at least 70%, at least 80% or at least 90%, greater, or at least 0.5 logs or at least 1.0 log higher than the level of let-7b-5p miRNA in the control individual. In some aspects, the let-7b-5p miRNA may be circulating let-7b-5p miRNA. In some aspects, the let-7b-5p miRNA may be EV-associated let-7b-5p miRNA. In some aspects, the let-7b-5p miRNA may comprise circulating let-7b-5p miRNA and EV-associated let-7b-5p miRNA.

In some aspects, the cancer-dependent analyte comprises miR-184 miRNA, and the individual is identified as having lung cancer if the level of miR-184 miRNA in the test individual is significantly less than the level of miR-184 miRNA in the control individual. In some aspects, the cancer-dependent analyte comprises miR-184 miRNA, and the individual is identified as having lung cancer if the level of miR-184 miRNA in the test individual is at least 20%, at least 30%, at least 40%, at least 50%, at least 60%, at least 70%, at least 80% or at least 90% lower than the level of miR-184 miRNA in the control individual. In some aspects, the cancer-dependent analyte comprises miR-184 miRNA, and the individual is identified as having lung cancer if the level of miR-184 miRNA in the test individual is at least 0.5 logs or at least 1.0 log lower than the level of miR-184 miRNA in the control individual. In some aspects, the miR-184 miRNA may be circulating miR-184 miRNA. In some aspects, the miR-184 miRNA may be EV-associated miR-184 miRNA. In some aspects, the miR-184 miRNA may comprise circulating miR-184 miRNA and EV-associated miR-184 miRNA.

In some aspects, the cancer-dependent analyte comprises miR-22-3p miRNA, and the individual is identified as having lung cancer if the level of miR-22-3p miRNA in the test individual is significantly less than the level of miR-22-3p miRNA in the control individual. In some aspects, the cancer-dependent analyte comprises miR-22-3p miRNA, and the individual is identified as having lung cancer if the level of miR-22-3p miRNA in the test individual is at least 20%, at least 30%, at least 40%, at least 50%, at least 60%, at least 70%, at least 80% or at least 90% lower than the level than the level of miR-22-3p miRNA in the control individual. In some aspects, the cancer-dependent analyte comprises miR-22-3p miRNA, and the individual is identified as having lung cancer if the level of miR-22-3p miRNA in the test individual is at least 0.5 logs or at least 1.0 log lower than the level of miR-22-3p miRNA in the control individual. In some aspects, the miR-22-3p miRNA may be circulating miR-22-3p miRNA. In some aspects, the miR-22-3p miRNA may be EV-associated miR-22-3p miRNA. In some aspects, the miR-22-3p miRNA may comprise circulating miR-22-3p miRNA and EV-associated miR-22-3p miRNA.

One aspect of the disclosure is a method of identifying a test individual as having lung cancer, comprising comparing the level of more than one analyte in the test individual with the level of the corresponding more than one analyte in a control individual, wherein if the level of one or more of the analytes in the test individual significantly differs from the level of the corresponding one or more analytes in the control individual, identifying the teste individual as having lung cancer. In some aspects, the method comprises:
i) comparing the level of let-7b-5p miRNA in the control individual with the level of let-7b-5p miRNA in a control individual;
ii) comparing the level of miR-184 miRNA in the control individual with the level of miR-184 miRNA in a control individual; and,

iii) comparing the level of miR-22-3p miRNA in the control individual with the level of miR-22-3p miRNA in a control individual;
wherein if the level of let-7b-5p miRNA in the test individual is significantly greater than the level of let-7b-5p miRNA in the control individual; and,
the level of miR-184 miRNA in the test individual is significantly less than the level of miR-184 miRNA in the control individual; and,
the level of miR-22-3p miRNA in the test individual is significantly less than the level of miR-22-3p miRNA in the control individual;
identifying the individual as having lung cancer. In some aspects, the let-7b-5p miRNA, the miR-184 miRNA, and/or the miR-22-3p miRNA may be circulating miRNA. In some aspects, let-7b-5p miRNA, the miR-184 miRNA, and/or the miR-22-3p miRNA may be EV-associated miRNA. In some aspects, let-7b-5p miRNA, the miR-184 miRNA, and/or the miR-22-3p miRNA may comprise circulating miRNA and EV-associated miRNA. In some aspects, the let-7b-5p miRNA and the miR-184 miRNA may be EV-associated miRNA, and the miR-22-3pp miRNA may be circulating miRNA. In some aspects, the level of miR-184 miRNA in the test individual is considered significantly less than the level of miR-184 miRNA in the control individual, if the level of miR-184 miRNA in the test individual is at least 20%, at least 30%, at least 40%, at least 50%, at least 60%, at least 70%, at least 80% or at least 90% lower than the level of miR-184 miRNA in the control individual. In some aspects, the level of miR-184 miRNA in the test individual is considered significantly less than the level of miR-184 miRNA in the control individual, if the level of miR-184 miRNA in the test individual is at least 0.5 logs or at least 1.0 log lower than the level of miR-184 miRNA in the control individual. In some aspects, the level of miR-22-3p miRNA in the test individual is considered significantly less than the level of miR-22-3p miRNA in the control individual, if the level of miR-22-3p miRNA in the test individual is at least 20%, at least 30%, at least 40%, at least 50%, at least 60%, at least 70%, at least 80% or at least 90% lower than the level of miR-22-3p miRNA in the control individual. In some aspects, the level of miR-22-3p miRNA in the test individual is considered significantly less than the level of miR-22-3p miRNA in the control individual, if the level of miR-22-3p miRNA in the test individual is at least 0.5 logs lower than the level of miR-22-3p miRNA in the control individual. In some aspects, the level of let-7b-5p miRNA in the test individual is considered significantly greater than the level of let-7b-5p miRNA in the control individual, if the level of let-7b-5p miRNA in the test individual is at least 2-fold, at least 3-fold, at least 4-fold, or at least 5-fold higher than the level of let-7b-5p miRNA in the control individual. In some aspects, the level of let-7b-5p miRNA in the test individual is considered significantly greater than the level of let-7b-5p miRNA in the control individual, if the level of let-7b-5p miRNA in the test individual is at least 20%, at least 30%, at least 40%, at least 50%, at least 60%, at least 70%, at least 80%or at least 90%, greater, or at least 0.5 logs, or at least 1.0 log. higher than the level of let-7b-5p miRNA in the control individual. In some aspects, the control individual is the same as the test individual, but the control ratio is determined at a time when the individual was known to be free of lung cancer.

One aspect of the disclosure is a method of identifying a lung cancer patient as having a shortened survival time, the method comprising comparing the level of a cancer-dependent analyte in the test individual with the level the cancer-dependent analyte in the control individual, wherein if the level of the cancer-dependent analyte in the test individual is significantly less than the level of the cancer-dependent analyte in the control individual, identifying the individual as having a shortened survival time; and wherein the cancer-dependent analyte is selected from the group consisting of miR-184 miRNA and miR-22-3p miRNA. In some aspects, the method comprises comparing the level of miR-184 miRNA in the test individual with the level miR-184 miRNA in the control individual; and/or comparing the level of miR-22-3p miRNA in the test individual with the level miR-22-3p miRNA in the control individual; wherein if the level of miR-184 miRNA in the test individual is significantly less than the level of miR-184 miRNA in the control individual; and/or if the level of miR-22-3p miRNA in the test individual is significantly less than the level of miR-22-3p miRNA in the control individual; identifying the individual as having a shortened survival time. In some aspects, the miR-184 miRNA, and/or the miR-22-3p miRNA may be circulating miRNA. In some aspects, the miR-184 miRNA, and/or the miR-22-3p miRNA may be EV-associated miRNA. In some aspects, the miR-184 miRNA, and/or the miR-22-3p miRNA may comprise circulating miRNA and EV-associated miRNA. In some aspects, the miR-184 miRNA may be EV-associated miRNA, and the miR-22-3pp miRNA may be circulating miRNA. In some aspects, the level of miR-184 miRNA in the test individual is considered significantly less than the level of miR-184 miRNA in the control individual, if the level of miR-184 miRNA in the test individual is at least 20%, at least 30%, at least 40%, at least 50%, at least 60%, at least 70%, at least 80%or at least 90%lower than the level than the level of miR-184 miRNA in the control individual. In some aspects, the level of miR-184 miRNA in the test individual is considered significantly less than the level of miR-184 miRNA in the control individual, if the level of miR-184 miRNA in the test individual is at least 0.5 logs, or at least one log, lower than the level of miR-184 miRNA in the control individual. In some aspects, the level of miR-22-3p miRNA in the test individual is considered significantly less than the level of miR-22-3p miRNA in the control individual, if the level of miR-22-3p miRNA in the test individual is at least 20%, at least 30%, at least 40%, at least 50%, at least 60%, at least 70%, at least 80% or at least 90% lower than the level than the level of miR-22-3p miRNA in the control individual. In some aspects, the level of miR-22-3p miRNA in the test individual is considered significantly less than the level of miR-22-3p miRNA in the control individual, if the level of miR-22-3p miRNA in the test individual is at least 0.5 logs, or at least one log, lower than the level of miR-22-3p miRNA in the control individual. In some aspects, the control individual is the same as the test individual, but the control ratio is determined at a time when the individual was known to be free of lung cancer.

One aspect of the disclosure is a method of identifying an individual having lung cancer as having stage IV lung cancer, comparing the level of a cancer-dependent analyte selected from the group consisting of CD63 and CD9 in the test individual, with the level of the cancer-dependent analyte in a control individual, wherein if the level of the cancer-dependent analyte in the test individual is significantly greater than the level of the cancer-dependent analyte in the control individual, identifying the individual as having stage IV lung cancer. In some aspects, the method may comprise comparing the level of CD63 or CD9 in the test individual with the level of a cancer-independent analyte in the test individual to produce a test ratio (e.g., a CD63/cancer-independent analyte test ratio or a CD9/cancer-independent analyte test ratio), and using the test ratio to identify the individual as having stage IV lung cancer. In some aspects, the cancer-dependent analyte may be CD63 and the test individual is identified as having stage IV lung cancer when the CD63/cancer-independent analyte test ratio is at least 1.0, at least 1.5, at least 2.0 at least 3.0, or at least 4.0. In some aspects, the cancer-dependent analyte may be CD9 and the test individual is identified as having stage IV lung cancer when the CD9/cancer-independent analyte test ratio in the test individual is at least 1.5, at least 2.0, at least 2.5, or at least 3.0. In some aspects, the cancer-independent analyte may be flotillin. Thus, in some aspects, the test individual is identified as having stage IV lung cancer when the CD63/flotillin test ratio in the test individual is greater than at least 1.0, at least 1.5, at least 2.0 at least 3.0, or at least 4.0. In some aspects, the test individual is identified as having stage IV lung cancer when the CD9/flotillin test ratio in the test individual is at least 1.5, at least 2.0, at least 2.5, or at least 3.0. In some aspects, the control individual is the same as the test individual, but the control ratio is determined at a time when the individual was known to be free of lung cancer.

In some aspects, the method may comprise comparing the test ratio from the individual with a control ratio produced using the levels of the same analytes used to produce the test ratio, but from a control individual. Thus, the CD63/cancer-independent analyte test ratio may be compared with a CD63/cancer-independent analyte control ratio, and the comparison used to identify the individual as having stage IV lung cancer; or the CD9/cancer-independent analyte test ratio may be compared with a CD9/cancer-independent analyte control ratio, and the comparison used to identify the individual as having stage IV lung cancer. In some aspects, the individual is identified as having stage IV lung cancer if the test ratio is significantly larger than the control ratio. In some aspects, the cancer-dependent analyte may be CD63, and the test individual is identified as having stage IV lung cancer when the ratio of the CD63/cancer-independent analyte test ratio to the CD63/cancer-independent analyte control ratio is at least 2.0, at least 2.5, at least 3.0, at least 3.5, at least 4.0, at last 4.5 at least 5.0, or at least 6.0. In some aspects, the cancer-dependent analyte may be CD9, and the test individual is identified as having stage IV lung cancer when the ratio of the CD9/cancer-independent analyte test ratio to the CD9/cancer-independent analyte control ratio is at least 1.5 or at least 2.0. In some aspects, the cancer-independent analyte may be flotillin, and the test individual may be identified as having stage IV lung cancer if the test ratio is significantly larger than the control ratio. In some aspects, the cancer-dependent analyte may be CD63, the cancer-independent analyte may be flotillin, and the test individual is identified as having stage IV lung cancer when the ratio of the CD63/flotillin test ratio to the CD63/flotillin control ratio is at least 2.0, at least 2.5, at least 3.0, at least 3.5, at least 4.0, at last 4.5 at least 5.0, or at least 6.0. In some aspects, the cancer-dependent analyte may be CD9, the cancer-independent analyte may be flotillin, and the test individual is identified as having stage IV lung cancer when the ratio of the CD63/flotillin test ratio to the CD63/flotillin control ratio is at least 1.5 or at least 2.0. In some aspects, the CD63 or CD9 may be circulating CD63 or circulating CD9. In some aspects, the CD63 or CD9 may be EV-associated CD63 or EV-associated CD9. In some aspects, the CD63 may comprise circulating CD63 and EV-associated CD63. In some aspects, the CD9 may comprise circulating CD9 and EV-associated CD9. In some aspects, the cancer-independent analyte may be circulating cancer-independent analyte. In some aspects, the cancer-independent analyte may be EV-associated cancer-independent analyte. In some aspects, the cancer-independent analyte may comprise circulating cancer-independent analyte and EV-associated cancer-independent analyte. In some aspects, the control individual is the same as the test individual, but the control ratio is determined at a time when the individual was known to be free of lung cancer.

One aspect of the disclosure is a method of identifying a test individual as having stage IV lung cancer, comprising comparing the level of a first cancer-dependent analyte in the test individual, with the level of a second cancer-dependent analyte in the test individual, there by obtaining a test ratio; and, using the test ratio to identify the individual as having stage IV lung cancer. In some aspects, the first cancer-dependent analyte may be CD9 and the second cancer-dependent analyte may be CD63, and the test individual is identified as having stage IV lung cancer when the CD9/CD63 test ratio in the test individual is less than 2.0. In some aspects, the first cancer-dependent analyte may be CD9, and the second cancer-dependent analyte may be CD63, and the test individual is identified as having stage IV lung cancer when the CD9/CD63 test ratio in the test individual is less than 1.5. In some aspects, the first cancer-dependent analyte may be CD9, and the second cancer-dependent analyte may be CD63, and the test individual is identified as having stage IV lung cancer when the CD9/CD63 test ratio in the test individual is less than 1.0. In some aspects, the first cancer-dependent analyte may be CD9, and the second cancer-dependent analyte may be CD63, and the test individual is identified as having stage IV lung cancer when the CD9/CD63 test ratio in the test individual is less than 0.5. In some aspects, the first cancer-dependent analyte and/or the second cancer-dependent analyte may be circulating analytes. In some aspects, the first cancer-dependent analyte and/or the second cancer-dependent analyte may be EV-associated analytes. In some aspects, the first cancer-dependent analyte and/or the second cancer-dependent analyte may comprise circulating analyte and EV-associated analyte. In some aspects, the first cancer-dependent analyte may be EV-associated CD9 (EV CD9), and the second cancer-dependent analyte may be EV-associated CD63 (EV CD63), and the test individual is identified as having lung cancer when the EV CD9/EV CD63 ratio in the test individual is less than 2.0, less than 1.5, less than 1.0, or less than 0.5. In some aspects, the control individual is the same as the test individual, but the control ratio is determined at a time when the individual was known to be free of lung cancer.

In some aspects, the method may comprise comparing the test ratio from the individual with a control ratio produced using the levels of the same analytes used to produce the test ratio, but from a control individual. Thus, the cancer-dependent analyte/cancer-independent analyte test ratio may be compared with a cancer-dependent analyte/cancer-independent analyte control ratio, and the comparison used to identify the individual as having stage IV lung cancer. In some aspects, the individual is identified as having stage IV lung cancer if the test ratio is significantly larger than the control ratio. In some aspects, the individual is identified as having stage IV lung cancer if the control ratio is significantly smaller than the test ratio. In some aspects, the first cancer-dependent analyte may be CD9, and the second cancer-dependent analyte may be CD63, and the test individual is identified as having stage IV lung cancer when the ratio of the test ratio to the control ratio is at least 4.0, at least 5.0, at least 6.0, at least 7.0, or at least 8.0. In some aspects, the first cancer-dependent analyte may be EV CD9, and the second cancer-dependent analyte may be EV CD63, and the test individual is identified as having stage IV lung cancer when the ratio of the test ratio to the control ratio is at least 4.0, at least 5.0, at least 6.0, at least 7.0, or at least 8.0. In some aspects, the control individual is the same as the test individual, but the control ratio is determined at a time when the individual was known to be free of lung cancer.

One aspect of the disclosure is a method of identifying an individual having lung cancer as having stage IV lung cancer, comprising comparing the level of a cancer-dependent analyte in the test individual, with the level of the cancer-dependent analyte in a control individual, and identifying the individual as having lung cancer if the level of the cancer-dependent analyte in the test individual is significantly different than the level of the cancer-dependent analyte in the control individual, wherein the cancer-dependent analyte comprises an miRNA selected from the group consisting of has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p. In some aspects, the miRNA comprises a circulating miRNA. In some aspects, the miRNA comprises an EV-associated miRNA. In some aspects, the miRNA comprises a circulating miRNA and an EV-associated miRNA. In some aspects, the control individual is the same as the test individual, but the control ratio is determined at a time when the individual was known to be free of lung cancer.

In some aspects, the cancer-dependent analyte may comprise let-7b-5p miRNA, and the individual is identified as having lung cancer if the level of let-7b-5p miRNA in the test individual is significantly greater than the level of let-7b-5p miRNA in the control individual. In some aspects, the cancer-dependent analyte may comprise let-7b-5p miRNA, and the individual is identified as having lung cancer if the level of let-7b-5p miRNA in the test individual is at least 2×, at least 3×, at least 4×, or at least 5× higher than the level of let-7b-5p miRNA in the control individual. In some aspects, the cancer-dependent analyte may comprise let-7b-5p miRNA, and the individual is identified as having lung cancer if the level of let-7b-5p miRNA in the test individual is at least 20%, at least 30%, at least 40%, at least 50%, at least 60%, at least 70%, at least 80% or at least 90%, greater, or at least 0.5 logs or at least 1.0 log higher, than the level of let-7b-5p miRNA in the control individual. In some aspects, the let-7b-5p miRNA may be circulating let-7b-5p miRNA. In some aspects, the let-7b-5p miRNA may be EV-associated let-7b-5p miRNA. In some aspects, the let-7b-5p miRNA may comprise circulating let-7b-5p miRNA and EV-associated let-7b-5p miRNA.

In some aspects, the cancer-dependent analyte may comprise miR-184 miRNA, and the individual is identified as having lung cancer if the level of miR-184 miRNA in the test individual is significantly less than the level of miR-184 miRNA in the control individual. In some aspects, the cancer-dependent analyte may comprise miR-184 miRNA, and the individual is identified as having lung cancer if the level of miR-184 miRNA in the test individual is at least 20%, at least 30%, at least 40%, at least 50%, at least 60%, at least 70%, at least 80% or at least 90% lower than the level than the level of miR-184 miRNA in the control individual. In some aspects, the cancer-dependent analyte may comprise miR-184 miRNA, and the individual is identified as having lung cancer if the level of miR-184 miRNA in the test individual is at least 0.5 logs or at least 1.0 log lower than the level of miR-184 miRNA in the control individual. In some aspects, the miR-184 miRNA may be circulating miR-184 miRNA. In some aspects, the miR-184 miRNA may be EV-associated miR-184 miRNA. In some aspects, the miR-184 miRNA may comprise circulating miR-184 miRNA and EV-associated miR-184 miRNA.

In some aspects, the cancer-dependent analyte may comprise miR-22-3p miRNA, and the individual is identified as having lung cancer if the level of miR-22-3p miRNA in the test individual is significantly less than the level of miR-22-3p miRNA in the control individual. In some aspects, the cancer-dependent analyte may comprise miR-22-3p miRNA, and the individual is identified as having lung cancer if the level of miR-22-3p miRNA in the test individual is at least 20%, at least 30%, at least 40%, at least 50%, at least 60%, at least 70%, at least 80%or at least 90%lower than the level than the level of miR-22-3p miRNA in the control individual. In some aspects, the cancer-dependent analyte may comprise miR-22-3p miRNA, and the individual is identified as having lung cancer if the level of miR-22-3p miRNA in the test individual is at least 0.5 logs or at least 1.0 log lower than the level of miR-22-3p miRNA in the control individual. In some aspects, the miR-22-3p miRNA may be circulating miR-22-3p miRNA. In some aspects, the miR-22-3p miRNA may be EV-associated miR-22-3p miRNA. In some aspects, the miR-22-3p miRNA may comprise circulating miR-22-3p miRNA and EV-associated miR-22-3p miRNA.

In some aspects, the method may comprise:
i) comparing the level of let-7b-5p miRNA in the control individual with the level of let-7b-5p miRNA in a control individual;
ii) comparing the level of miR-184 miRNA in the control individual with the level of miR-184 miRNA in a control individual; and,
iii) comparing the level of miR-22-3p miRNA in the control individual with the level of miR-22-3p miRNA in a control individual;
wherein if the level of let-7b-5p miRNA in the test individual is significantly greater than the level of let-7b-5p miRNA in the control individual; and,
the level of miR-184 miRNA in the test individual is significantly less than the level of miR-184 miRNA in the control individual; and,
the level of miR-22-3p miRNA in the test individual is significantly less than the level of miR-22-3p miRNA in the control individual;
identifying the individual as having lung cancer. In some aspects, the let-7b-5p miRNA, the miR-184 miRNA, and/or the miR-22-3p miRNA may be circulating miRNA. In some aspects, let-7b-5p miRNA, the miR-184 miRNA, and/or the miR-22-3p miRNA may be EV-associated miRNA. In some aspects, let-7b-5p miRNA, the miR-184 miRNA, and/or the miR-22-3p miRNA may comprise circulating miRNA and EV-associated miRNA. In some aspects, the let-7b-5p miRNA and the miR-184 miRNA may be EV-associated miRNA, and the miR-22-3pp miRNA may be circulating miRNA. In some aspects, the level of miR-184 miRNA in the test individual is considered significantly less than the level of miR-184 miRNA in the control individual, if the level of miR-184 miRNA in the test individual is at least 20%, at least 30%, at least 40%, at least 50%, at least 60%, at least 70%, at least 80% or at least 90% lower than the level of miR-184 miRNA in the control individual. In some aspects, the level of miR-184 miRNA in the test individual is considered significantly less than the level of miR-184 miRNA in the control individual, if the level of miR-184 miRNA in the test individual is at least 0.5 logs or at least 1.0 log lower than the level of miR-184 miRNA in the control individual. In some aspects, the level of miR-22-3p miRNA in the test individual is considered significantly less than the level of miR-22-3p miRNA in the control individual, if the level of miR-22-3p miRNA in the test individual is at least 20%, at least 30%, at least 40%, at least 50%, at least 60%, at least 70%, at least 80% or at least 90% lower than the level of miR-22-3p miRNA in the control individual. In some aspects, the level of miR-22-3p miRNA in the test individual is considered significantly less than the level of miR-22-3p miRNA in the control individual, if the level of miR-22-3p miRNA in the test individual is at least 0.5 logs lower than the level of miR-22-3p miRNA in the control individual. In some aspects, the level of let-7b-5p miRNA in the test individual is considered significantly greater than the level of let-7b-5p miRNA in the control individual, if the level of let-7b-5p miRNA in the test individual is at least 2×, at least 3×, at least 4×, or at least 5× higher than the level of let-7b-5p miRNA in the control individual. In some aspects, the level of let-7b-5p miRNA in the test individual is considered significantly greater than the level of let-7b-5p miRNA in the control individual, if the level of let-7b-5p miRNA in the test individual is at least 20%, at least 30%, at least 40%, at least 50%, at least 60%, at least 70%, at least 80% or at least 90%, greater, or at least 0.5 logs higher than the level of let-7b-5p miRNA in the control individual.

One aspect of the disclosure is a method of identifying a lung cancer patient having drug-resistant cancer, the method comprising comparing the level of a cancer-dependent analyte in the test individual with the level the cancer-dependent analyte in the control individual; wherein if the level of the cancer-dependent analyte in the test individual is significantly less than the level of the cancer-dependent analyte in the control individual; identifying the individual as having a drug-resistant lung tumor; and wherein the cancer-dependent analyte is selected from the group consisting of miR-184 miRNA and miR-22-3p miRNA. In some aspects, the method comprises comparing the level of miR-184 miRNA in the test individual with the level miR-184 miRNA in the control individual; and/or comparing the level of miR-22-3p miRNA in the test individual with the level miR-22-3p miRNA in the control individual; wherein if the level of miR-184 miRNA in the test individual is significantly less than the level of miR-184 miRNA in the control individual; and/or if the level of miR-22-3p miRNA in the test individual is significantly less than the level of miR-22-3p miRNA in the control individual; identifying the individual as having a drug-resistant lung tumor. In some aspects, the miR-184 miRNA, and/or the miR-22-3p miRNA may be circulating miRNA. In some aspects, the miR-184 miRNA, and/or the miR-22-3p miRNA may be EV-associated miRNA. In some aspects, the miR-184 miRNA, and/or the miR-22-3p miRNA may comprise circulating miRNA and EV-associated miRNA. In some aspects, the miR-184 miRNA may be EV-associated miRNA, and the miR-22-3pp miRNA may be circulating miRNA. In some aspects, the level of miR-184 miRNA in the test individual is considered significantly less than the level of miR-184 miRNA in the control individual, if the level of miR-184 miRNA in the test individual is at least 2×, at least 3×, at least 4×, or at least 5× lower than the level of miR-184 miRNA in the control individual. In some aspects, the level of miR-184 miRNA in the test individual is considered significantly less than the level of miR-184 miRNA in the control individual, if the level of miR-184 miRNA in the test individual is at least 0.5 logs lower than the level of miR-184 miRNA in the control individual. In some aspects, the level of miR-22-3p miRNA in the test individual is considered significantly less than the level of miR-22-3p miRNA in the control individual, if the level of miR-22-3p miRNA in the test individual is at least 2×, at least 3×, at least 4×, or at least 5× lower than the level of miR-22-3p miRNA in the control individual. In some aspects, the level of miR-22-3p miRNA in the test individual is considered significantly less than the level of miR-22-3p miRNA in the control individual, if the level of miR-22-3p miRNA in the test individual is at least 0.5 logs lower than the level of miR-22-3p miRNA in the control individual. In some aspects, the control individual is the same as the test individual, but the control ratio is determined at a time when the individual was known to be free of lung cancer.

One aspect of the disclosure is a method of decreasing the treatment-resistance of a lung cancer in a cancer patient, comprising modulating the expression of let-7b-5p miRNA, miR-184 miRNA, and/or miR-22-3p miRNA in the cancer patient. In some aspects, the method comprises decreasing the level of let-7b-5p miRNA in the cancer patient. In some aspects, the method comprises increasing the level of miR-184 miRNA and/or miR-22-3p miRNA in the cancer patient. In some aspects, increasing the level of miR-184 miRNA and/or miR-22-3p miRNA in the cancer patient comprise administering to the cancer patient miR-184 miRNA and/or miR-22-3p miRNA, or a vector encoding miR-184 miRNA and/or miR-22-3p miRNA.

One aspect of the disclosure is a method of sensitizing lung cancer cells to treatment in a cancer patient, comprising modulating the expression of let-7b-5p miRNA, miR-184 miRNA, and/or miR-22-3p miRNA in the cancer patient. In some aspects, the method comprises decreasing the level of let-7b-5p miRNA in the cancer patient. In some aspects, the method comprises increasing the level of miR-184 miRNA and/or miR-22-3p miRNA in the cancer patient. In some aspects, increasing the level of miR-184 miRNA and/or miR-22-3p miRNA in the cancer patient comprise administering to the cancer patient miR-184 miRNA and/or miR-22-3p miRNA, or a vector encoding miR-184 miRNA and/or miR-22-3p miRNA.

One aspect of the disclosure is a method of treating an individual for lung cancer, comprising comparing the level of miR-184 miRNA in the test individual with the level miR-184 miRNA in the control individual; and/or comparing the level of miR-22-3p miRNA in the test individual with the level miR-22-3p miRNA in the control individual; wherein if the level of miR-184 miRNA in the test individual is significantly less than the level of miR-184 miRNA in the control individual; and/or if the level of miR-22-3p miRNA in the test individual is significantly less than the level of miR-22-3p miRNA in the control individual; administering to the individual a tyrosine kinase inhibitor and an inhibitor of one or more proteins in the p13k/Akt pathway. In some aspects, the tyrosine kinase inhibitor is selected from the group consisting of Erlotinib, Afatinib, Gefiiniband, Osimertinib, and combinations thereof. In some aspects, the control individual is the same as the test individual, but the control ratio is determined at a time when the individual was known to be free of lung cancer.

One aspect of the disclosure is a kit comprising:
i) reagents for use in determining the level of at least one cancer-dependent analyte; and,
ii) instructions for using the reagents to determine the level of the at least at least one cancer-dependent analyte;
wherein the cancer-dependent analyte is selected from the group consisting of CD9, CD63, has-let-7b-5p, has-miR-26a-5p, has-miR-122-5p, has-miR-200c-3p, has-miR-148a-3p, has-miR-378a-3p, has-miR-192-5p, has-miR-21-5p, has-miR-141-3p, has-miR-155-5p, has-miR-10b-5p, has-miR-184, has-miR-92b-3p, has-miR-574-5p, has-miR-203a-3p, has-miR-31-5p, has-miR-100-5p, has-miR-363-3p, has-miR-4454, has-miR-27a-5p, has-miR-4700-5p, has-miR-7155-5p, has-miR-127-3p, has-miR-29c-3p, has-miR-423-5p, has-miR-486-5p, has-miR-320a-3p, has-miR-185-5p, has-miR-99a-5p, has-miR-22-3p, has-miR-192-5p, has-miR-148a-3p, has-miR-21-5p, has-miR-143-3p, has-miR-184, has-miR-3976, has-miR-4259, has-miR-598-3p, has-miR-7-5p, has-miR-223-3p, has-miR-16-5p, has-miR-425-5p, has-miR-146a-5p, has-miR-92b-3p, has-let-7i-5p, has-miR-451a, has-miR-142-5p, and has-let-7c-5p. In some aspects, the at least one cancer-dependent analyte may be a circulating analyte. In some aspects, the at least one cancer-dependent analyte may be an EV-associated analyte. In some aspects, the at least one cancer-dependent analyte comprises a circulating analyte and an EV-associated analyte. In some aspects, the kit comprises instructions for identifying an individual having lung cancer, identifying an individual having a drug-resistant lung cancer, identifying an individual having a shortened survival time, treating an individual for lung cancer.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLES

### Materials and Methods

### Isolation of Extracellular Vesicles and Circulating Micro-RNA

Patients' blood was double-centrifuged to remove platelets and the platelet-poor plasma was used for EV analysis. Purification of the EVs was done through size exclusion chromatography (SEC) using qEV70s single columns (ddIzon, Netherlands). These columns were filled with 150 μl of plasma and used based on recommendations from the manufacturer; fraction 8 through 11 were collected and pooled to obtain the fraction of purified EVs. The particle size and concentration for all EV samples were determined using nanoparticle tracking analysis (NTA).

Total RNA extraction was performed using 200 μl of plasma or 200 μl of pooled EV fractions and the miRNeasy mini kit (Qiagen). Samples were thawed at room temperature followed by centrifugation at 12,000×g for 5 minutes at 4° C. to remove any debris. Extraction was conducted by use of the miRNA easy Qiagen kit. For homogenization, 200 μL of plasma/EV suspension were mixed with 1000 μL Qiazol and 1 μL of a mix of 3 synthetic spike-in controls (Qiagen, Germany). After a 10-minute incubation at room temperature, 200 μL chloroform were added to the lysates followed by cooled centrifugation at 12,000×g for 15 minutes at 4° C. Precisely 650 μL of the upper aqueous phase were mixed with 7 μL glycogen (50 mg/mL) to enhance precipitation. Samples were transferred to a miRNeasy mini column, and RNA was precipitated with 750 μL ethanol followed by automated washing with RPE and RWT buffer in a QiaCube liquid handling robot. Finally, total RNA was eluted in 30 μL nuclease free water and stored at −80° C. until further use.

### Electron Microscopy

Exosomes were processed for negative staining as described in M. Rames, et al. Briefly, 5 μL of purified EV sample was applied to a freshly glow discharged (Pelco Easiglow, Ted Pella Redding CA) carbon coated TEM grid (Electron Microscopy Sciences, Hatfield PA) over ice. Samples were washed three times with distilled water, incubated 2 min on 2% paraformaldehyde (Electron Microscopy Sciences), then incubated for 30 sec on 2% uranyl acetate (aqueous, Electron Microscopy Sciences) and back-blotted with filter paper (Whatman P1, Fisher Scientific) and allowed to dry. Images were collected on a JEOL JEM 1400 transmission electron microscope operated at 120V equipped with a Gatan Ultrascan 1000 CCD camera.

### Small RNA Sequencing

Equal volumes of total RNA (2 μL) were used for small RNA library preparation using the Clean Tag small RNA library preparation kit (TriLink Biotechnologies, US). that utilizes chemically modified adapters to prevent formation of adapter dimers. Adapter-ligated libraries were amplified using barcoded Illumina reverse primers in combination with the Illumina forward primer. A pool consisting of 40 plasma samples, and a second pool consisting of 40 EV samples was prepared by mixing samples at equimolar rates based on a DNA-1000 bioanalyzer results (Agilent, CA). The DNA library pool underwent size-selection (BluePippin, SageScience, US) to enrich for microRNAs with an insert size of 18-36 nt, corresponding to a library size of approximately 145 bp.

Sequencing was performed on an Illumina NextSeq 550 with 75 bp single end runs. Overall quality of the next-generation sequencing data was evaluated automatically and manually with FastQC v0.11.8 and MultiQC v1.7. Reads from all passing samples were adapter trimmed and quality filtered using Cutadapt v2.3 and filtered for a minimum length of 17nt. Mapping steps were performed with bowtie v1.2.2 and miRDeep2 v2.0.1.2, whereas reads were mapped first against the genomic reference GRCh38.p12 provided by Ensemble allowing for two mismatches and subsequently miRBase v22.1, filtered for miRNAs of hsa only, allowing for one mismatch. For a general RNA composition overview, non-miRNA mapped reads were mapped against RNAcentral and then assigned to various RNA species of interest.

Statistical analysis of preprocessed NGS data was done with R v3.6 and the packages pheatmap v1.0.12, pcaMethods v1.78 and genefilter v1.68. Differential expression analysis with edgeR v3.28 used the quasi-likelihood negative binomial generalized log-linear model (GLM) functions provided by the package. False discovery rate (FDR) correction was performed to adjust for multiple testing, and a cut-off of FDR<5% was applied.

### Target Network Analysis

miRNA target network analyses and genes ontology enrichments analyses (KEGG and Reactome) were conducted using miRNet (www.mirnet.ca).

### Cell Lines and Cell Culture

The human lung cancer cell line A549 were grown in Dulbecco's Modified Eagle Medium (DMEM, 11965-092), with L-Glutamine, and high glucose supplemented with 10% FBS. Cells were grown in the nutrient medium as suggested by ATCC. Cells were incubated in a humidified incubator with 5% CO2 at 37° C. H1975 cells were grown in ATCC-formulated RPMI-1640 Medium (ATCC, Cat number 30-2001), with 10% FBS (fetal bovine serum) at 37 C, 5% CO2.

### Peripheral Blood Mononuclear Cells (PBMC) Isolation

10 mL of blood from healthy individual was collected into EDTA coated anti-coagulant vacutainer tubes (BD Biosciences #367899). Transferred onto 50 mL sterile 50 mL centrifuge tube and added equal volume with of ice cold DPBS pH 7.4 and gently mix by inversion. Using transfer pipette, carefully transferred diluted blood to sterile centrifuge tube containing 10 mL of Ficoll-Paque plus (Amersham #17144003,) then centrifuge at 2000 rpm for 25 min. after centrifuged from the fractionated phases, carefully collect the PBMCs fraction between ficol-paque and plasma layer. Collected PBMCs washed with ice cold PBS and centrifuge at 1700 rpm for 10 min. pellet re-suspended with 2 ml of Pharm Lyse lysing buffer (Biosciences #555899), mix well incubate at 37° C. for 4 min and adjust volume with PBS to 50 mL and proceed with centrifuge at 1700 rpm for 10 min. PBMC pellet re suspend in PBS with 1×10⁶ cells/mL for further experimental purpose.

### EV/PBMC Supernatant Transfer Experiments

Isolated human PBMCs were seeded and grown in 6 well culture dish with hybridoma-(SFM) serum free medium (Gibco #12045076,). PBMCs were treated with patient EVs under serum free conditions for 24 hrs. After incubation cell free PBMC or EV/PBMC conditioned media were collected and used for culturing A549 cells. After 24 hours A549 cells were collected for cell counting or lysed for Western blotting.

### Western Blotting

Extracellular vesicles (EV). EV pellets were dissolved in 100 ul of modified lysis buffer (20 mM Tris-HCl pH-7.5, 150 mM NaCl, 1 mM Na₂EDTA, 1 mM EGTA, 1% TritonX-100, 2.5 mM sodium pyrophosphate, 1 mM b-glycerophosphate, 1 mM Na₃ VO₄, 1 μg/mL leupeptin). Samples were treated with 10% glycerol, 1 M urea, 0.1% SDS, and loading buffer before SDS-PAGE. Gels were transferred onto PVDF membranes and stained with primary antibodies against CD-9 (1:1000, Millipore #CBL162,), CD-63 (1:1000, Molecular probes #A15712), CD-81 (1:1000, BioLegend #349561), and flotillin (1:1000, Santa Cruz #74566). Secondary antibodies were anti-mouse horseradish peroxidase (1:10000, Invitrogen #31430). Protein bands were detected using the Pierce ECL Chemiluminescence kit western (Thermo Fisher #32106) and the ChemiDoc™ Imaging System, Bio-Rad Laboratories Inc.

EV-treated A549 cells. A549 cells were cultured with similar loads of EV derived either from high-risk controls or confirmed lung cancer patients. Lysates were prepared in lysis buffer (20 mM Tris-HCl pH-7.5, 150 mM NaCl, 1 mM Na₂ EDTA, 1 mM EGTA, 1% TritonX-100) and processed for Western blotting. Blots were stained against β-catenin to determine WNT signaling levels and GAPDH as a loading control (1:1000, Cell signaling #5174S). Secondary horseradish peroxidase (HRP) antibodies were obtained from Invitrogen. Pierce ECL Chemiluminescence kit (Thermo Fisher scientific #32106) and the ChemiDoc Imaging System (Bio-Rad) were used to detect protein bands.

A549 cells were washed with PBS and lysed in a lysis buffer (20 mM Tris-HCl pH-7.5, 150 mM, NaCl, 1 mM Na₂EDTA, 1 mM EGTA, 1% Triton, 2.5 mM sodium pyrophosphate, 1 mM b-glycerophosphate, 1 mM Na3VO4, 1 μg/ml leupeptin) supplemented with protease and a phosphatase inhibitor cocktail (Cell Signaling #9803S). Proteins were electrophoresed on SDS-PAGE using 4-20% Mini-PROTEAN® TGX™ precast gel (Biorad #456-1094,), transferred onto methanol pretreated PVDF membrane. PVDF membranes were probed overnight with mouse anti β-catenin (DSHB, #PY489), mouse anti-mTOR (1:1000, Santa Cruz, #517464), mouse anti phosphor-mTOR (1:1000, Santa Cruz, #293089), and mouse anti-GAPDH (DSHB, #2G7) at 4° C. Membranes were incubated with anti-rabbit mouse horseradish peroxidase (HRP) (1:5000, #31460, Invitrogen). Primary or secondary antibodies were diluted in 5% BSA-TBST. Anti-mouse horseradish peroxidase (HRP) (1:5000, Invitrogen #31430,), anti-rabbit horseradish peroxidase (HRP) (1:5000, Invitrogen #31460,) used to develop respective blots. Membranes were developed using Pierce ECL western blotting substrate (Thermo Fisher scientific #32106,) and imaged on ChemiDoc™ MP Imaging System, Bio-Rad Laboratories Inc.

H1975 cells. H1975 cells were lysed in 1× RIPA buffer (Thermofisher #89900) containing 1× Halt™ Protease Inhibitor Cocktail (Thermofisher #78425). Proteins were separated on a 4-12% Bis-Tris gel (Invitrogen, #NP0321) and transferred to PVDF membranes. Membranes were incubated with primary antibodies against AKT (1:1000, cell signaling #9272) or phospho-AKT (1:5000, Proteintech #66444-1-Ig,) or GAPDH (Sigma #G8795) overnight at 4° C. Secondary antibodies were purchased from Cell Signaling (#7076). Blots were developed with Immobilon Western Chemiluminescence Kit (Millipore, #WBKLS0500).

miRNA Transfections and Osimertinib treatments miRNA stock was prepared by suspending in RNAse-free water. Cells were seeded such that they were 70-80% confluent at the time of transfection. Cells were allowed to adhere for 24 hrs and then transfected with 25 nM of miRNA inhibitors against hsa-miR184 (Sigma #HSTUD0282), hsa-miR22-3p (Sigma #HSTUD0393) or 100 nM of hsa-let7b-5p miRNA mimic (Sigma c #HMI0007) using Lipofectamine 3000 (Invitrogen #L3000001). Osimertinib (100 nM) was added an hour after transfection to cells. Cells were allowed to incubate with transfection mix for 24 hrs at 37° C., 5% CO₂ and then washed with 1× PBS and trypsinized to be used for cell count and Western blotting.

### cDNA Synthesis and qRT-PCR

cDNA synthesis was carried out using the miRCURY LNA RT Kit (Qiagen #339340) from human purified EVs or plasma RNA (10 ng) according to manufacturer's instructions. The synthesized CDNA diluted in nuclease free water and stored until further use at −80° C. as per the kit instructions. Quantitative polymerase chain reaction was conducted using miRCURY LNA miRNA SYBR PCR kit (Qiagen #339345) as per manufacturer's instruction on BioRad CFX96™ System in 96-well plates in 3-6 repeats. A two-step thermal cycling protocol i.e., 95°° C. for 2 min followed by 40 cycles at 95° C. for 10 sec and 56° C. for 60 sec, was used. A no-reverse transcriptase (NRT) and no-template control (NTC) were included in each reaction to check for primer specificity and any non-specific amplification. miRNA targets include miRNA-184 (Qiagen #YP00204601), miRNA-21-5p (Qiagen #YP00204230), Let-7b-5p (Qiagen #YP00204750), miRNA 22-3p (Qiagen #YP00204606). The expression levels of each miRNA target were normalized to calibrators U6-snRNA or GAPDH. Fold changed of miRNA was calculated by ΔΔCt and 2^{-ΔΔCt} method. ΔCt was calculated by subtracting the average of Ct values of calibrator from Ct values of target miRNA. ΔΔCt was computed by subtracting ΔCt of the screening control from ΔCt of RADS IV group.

### Patient Data Analysis

MicroRNA expression (miRNAseq) and clinical data from Lung adenocarcinoma (LUAD) were collected from the publicly accessible TCGA database using the Bioconductor tool TCGA Biolinks RTCGA R packages. The “surv_cutpoint” function of the “survminer” R package was used to identify high versus low expressing patients' samples for survival analysis. Survminer uses selected rank statistics to determine the optimal cut-point of a continuous variable in an unbiased manner. Kaplan-Meier (KM) survival plots and related statistics were generated using the Survival R package.

### Leave-One-Out Cross Validation (LOOCV) Analyses

The robustness of the three predictor microRNAs (let-7b-5p, miR-184, miR-22-3p) for the binary outcome cancer-free versus confirmed cases were confirmed using complete let-7b-5p, miR-184, and miR-22-3p expression data for 35 samples. 35 regression models were generated, and each time left out one sample to build the model. The sample that was left out was classified using the model generated from the remaining 34 samples and the number of instances in which the prediction agreed with the actual outcome compared.

### Example 1. CD9 and CD63 Enrichment of Extracellular Vesicles From NSCLC Patients

This example demonstrates the identification of molecular signatures that discriminate between NSCLC patients and healthy individuals having a similar risk profile.

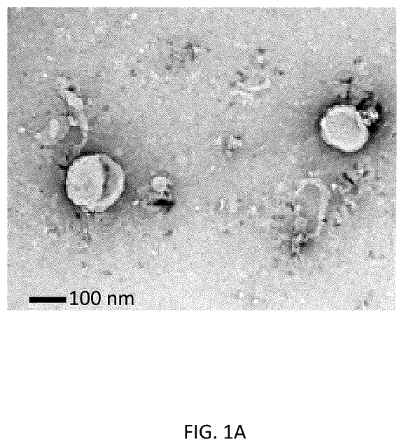

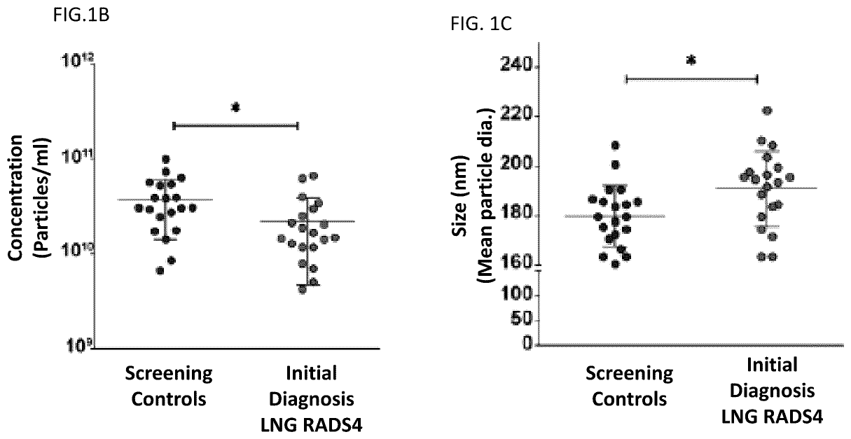

The US Preventive Services Task Force recommends LDCT screening for at-risk individuals defined as 55-80 years of age with 30 or more pack-year smoking history or have quit within the past 15 years. In this study, individuals ranging between 42 and 62 years of age with a qualifying smoking history were divided into two groups based on their initial LDCT Lung-RADS scores 2 or 4 (Table 1, Lung-RADS2 screening controls versus Lung-RADS4, N=20 per group). Plasma EVs were isolated from all 40 individuals (FIG. 1A) and nanoparticle tracking analyses (NTA) was performed to determine whether EV from Lung-RADS4 patients cohort exhibit physical characteristics that are distinct from control cohort EV. Initial analyses revealed that EV density was reduced in Lung-RADS4 patients compared to screening controls but that these EV were larger than Lung-RADS2 EV (FIGS. 1B & 1C).

Consistent with this, Follow up tissue biopsies analyses revealed that 30% (6 out of 20) of the patients who were classified as Lung-RADS4 at baseline LDCT screening did not have lung cancer (Table 1; group column, asterisk), which is consistent with the known false positive rate of 23-50% for LDCT. The density and size characteristics of the EV were analyzed to determine if such characteristics could differentiate between true Lung-RADS4 patients with lung cancer and the rest of the patients with benign lung nodules without cancers (Lung-RADS2 plus the false-positive Lung-RADS4 patients). Although the trend of reduced EV density and larger EV size in true Lung-RADS4 compared to controls persisted, these differences were not statistically significant (data not shown).

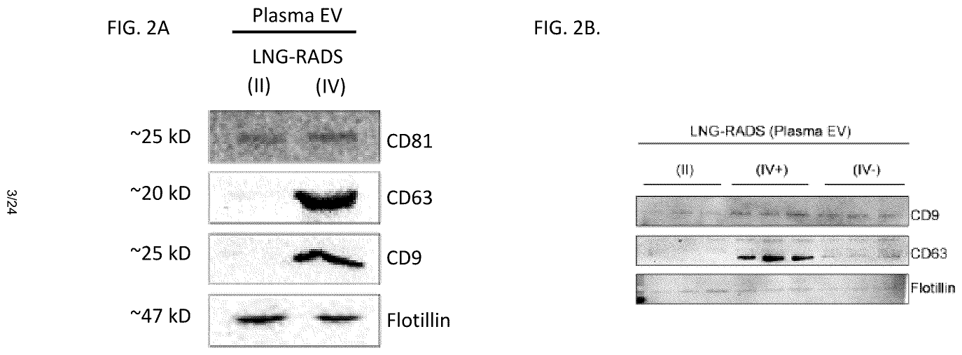

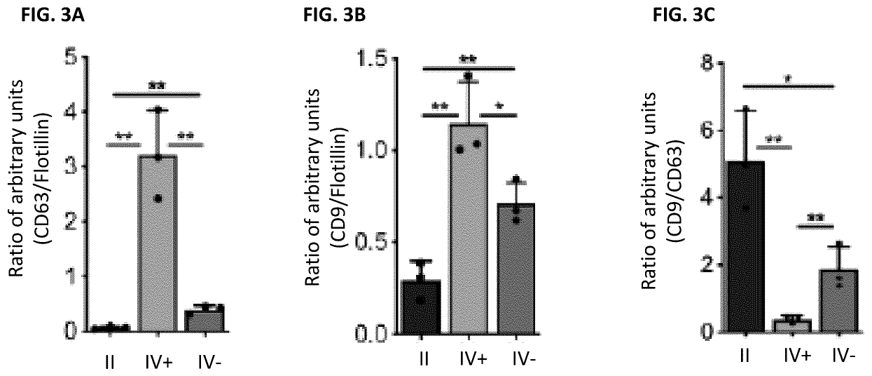

The molecular profiles of EV membranes were analyzed to identify differences between screening controls, over-diagnosed individuals, and confirmed cancer patients. Initial studies focused on the protein abundance of known EV membrane markers (the tetraspanins CD9, CD63, CD81, and the resident EV protein flotillin). While EV isolated from all Lung-RADS4 (initial diagnosis, including false-positive) patients and screening controls showed similar levels of CD81 and flotillin, CD63 and, to a lesser extent, CD9 were specifically enriched in Lung-RADS4 EV (CD63 high, CD9 moderate) compared to Lung-RADS2 screening controls (CD63 low and CD9 low) (FIG. 2A). EV exclusively derived from confirmed NSCLC patients (IV⁺) were CD63 high and CD9 moderate) when compared to EV from false-positive patients (CD63 low and CD9 moderate) or to screening controls (II: CD63 low and CD9 low) (FIGS. 2B, 3A and 3B). Consistent with these findings, the ratio of CD9/CD63 levels distinguished confirmed cases from false-positive Lung-RADS4 or screening individuals [FIG. 3C, (mean±SD): 0.37±0.1 versus 1.86±0.66 versus 5.08±1.48, respectively). These results demonstrate that EV CD9/CD63 expression ratio, but not EV physical characteristics, can differentiate NSCLC patients from cancer-free high-risk individuals.

### Example 2. Differentiation of NSCLC Patients From High-Risk Individuals

This example demonstrates the use of miRNA to differentiate NSCLC patients from high-risk individuals.

Next generation sequencing (NGS) was used to profile EV miRNA from Lung-RADS4 confirmed cancer patients or over-diagnosed Lung-RADS4 individuals or high-risk screening controls (Lung-RADS2). Circulating plasma miRNA was included because combining multiple analytes from diverse biological sources has the potential to identify robust biomarkers. 58 differentially expressed miRNAs were identified, including miRNA widely known to be deregulated in cancers. To identify a set of miRNAs that can robustly discriminate between NSCLC and cancer-free individuals, miRNAs that were differentially expressed in at least two of the following comparisons: Lung-RADS2 versus Lung-RADS4; Lung-RADS4 false positive versus confirmed cancer patients; Lung-RADS2 combined with false-positive Lung-RADS4 patients versus confirmed cancer patients; any of the preceding groups versus patients who rapidly progressed (LDCT imaging and/or death shortly after sampling), were prioritized. From this analysis, miRNAs showing significant performance (P and area under the curve/AUC values) in receiver operating characteristic (ROC) analyses were chosen for further examination. This approach led to the discovery of let-7b-5p, miR-184, and miR-22-3p as potential biomarkers for discriminating cancer patients from high-risk controls.

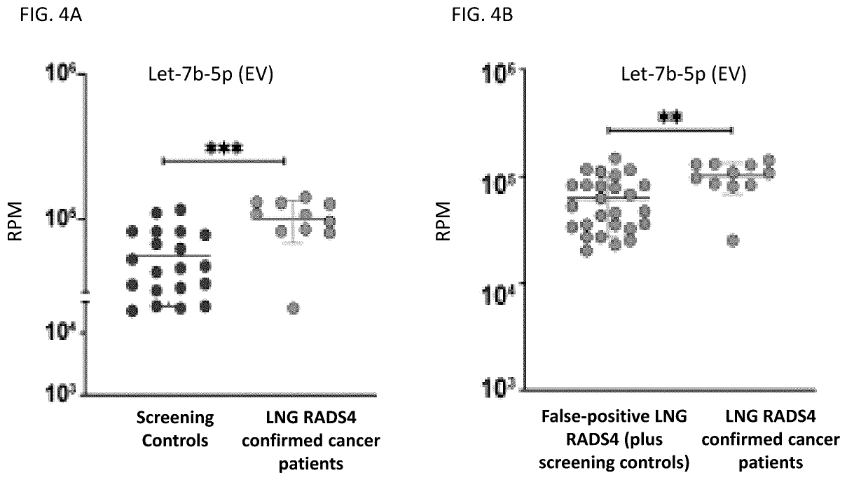

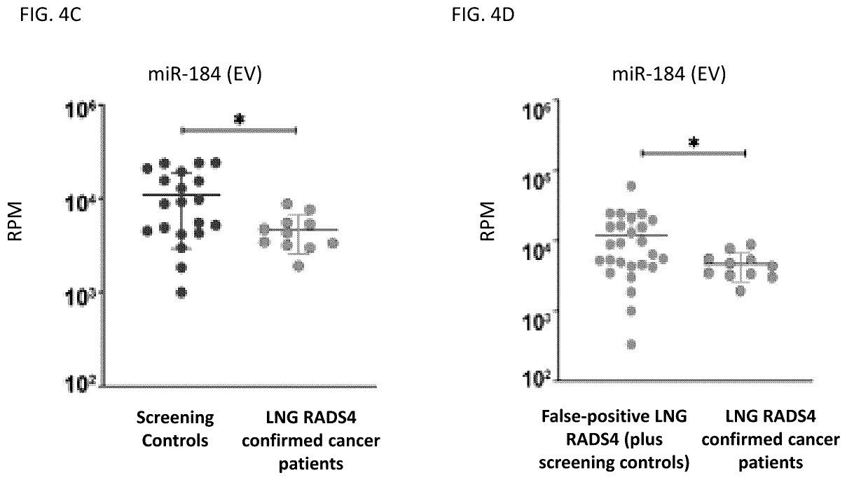

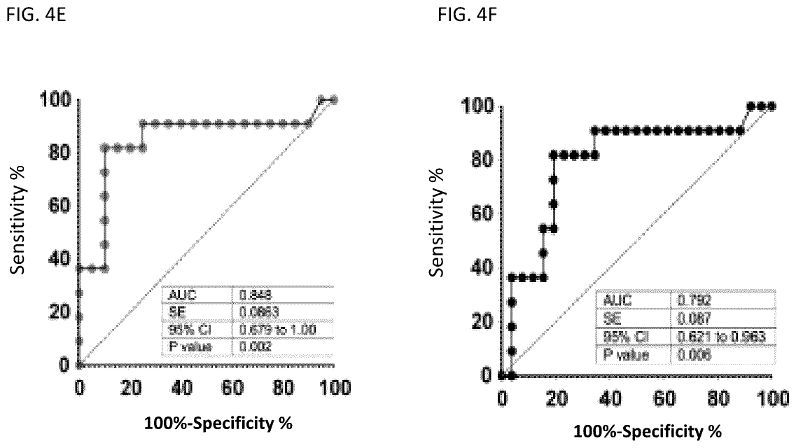

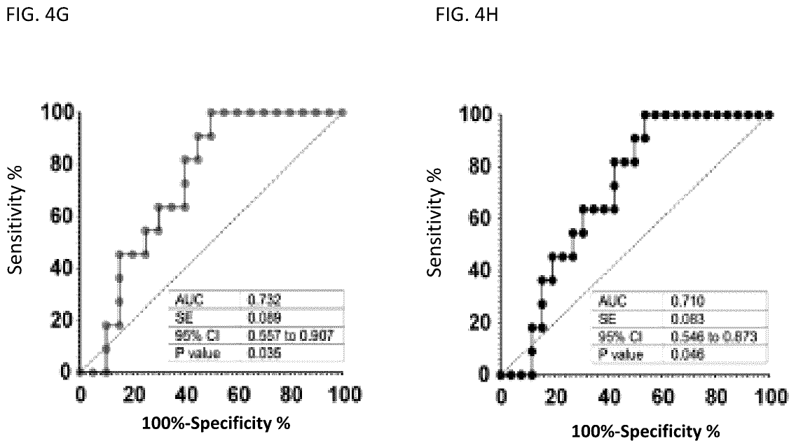

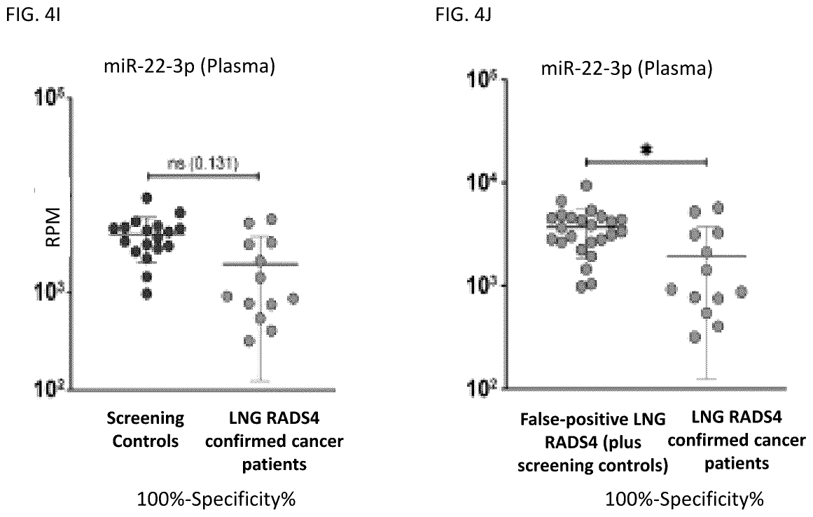

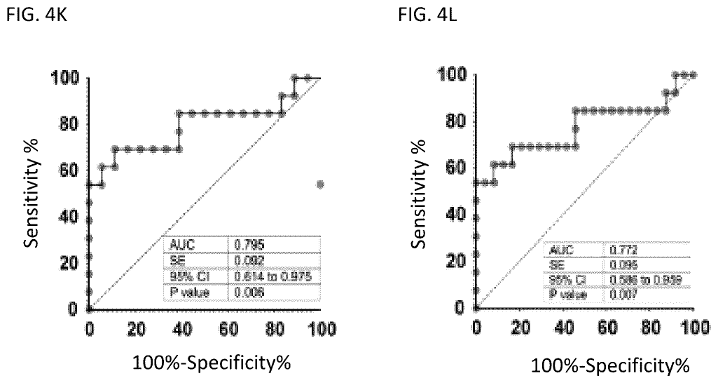

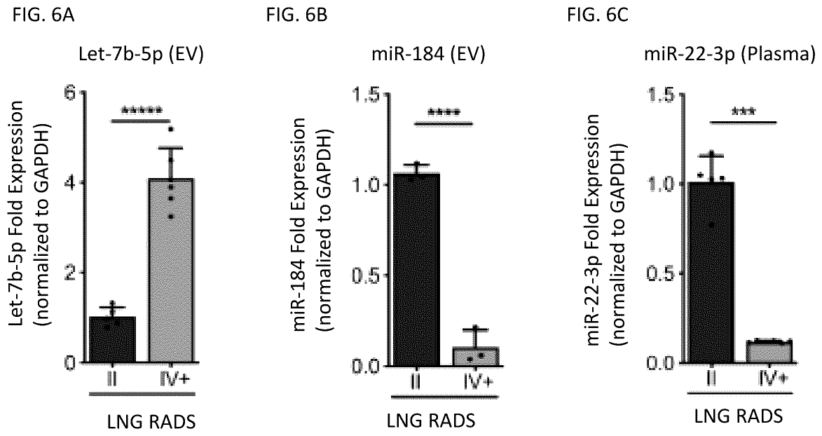

Analysis revealed that Let-7b-5p levels were elevated in EV from Lung-RADS4 patients compared to EV from screening patients alone (FIGS. 4A-4E) or to screening patients plus false-positive Lung-RADS4 patients combined (FIGS. 4B & 4F). Also, miR-184 abundance was significantly reduced in Lung-RADS4 EV compared to either control group (excluding or including false-positive Lung-RADS4 patients) (FIGS. 4C, 4D, 4G & 4H). Circulating miRNA analyses showed that miR-22-3p levels were reduced in Lung-RADS4 patients compared to either control group (FIGS. 4U-4L). ROC curves showed area under the curve (AUC) values above 70% and significant p-values for all the identified markers across comparisons (FIGS. 4E-4H, FIG. 4K and FIG. 4L). The expression of these miRNAs was independently verified in quantitative polymerase chain reaction (qPCR) experiments using EV or plasma samples obtained from controls or false-positives or confirmed cancer patients (FIGS. 6A-6C). Further, the expression of Let-7b-5p, miR-184, miR-22-3p showed no significant correlation with the gender, age, and pack-years smoking record of the participants), providing additional statistical rigor and indicating that the expression profile of these EV and circulating miRNAs is specifically associated with disease status.

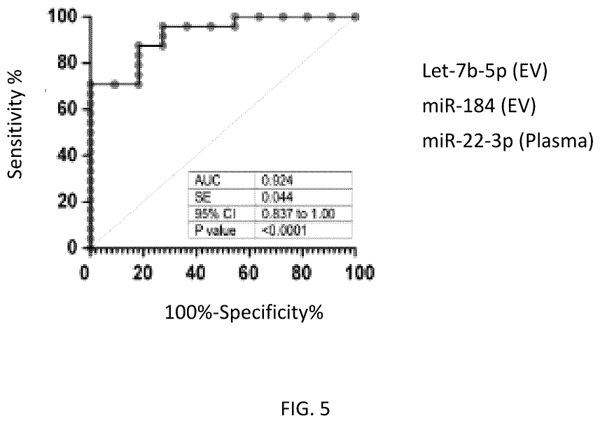

Moreover, multiple logistic regression analyses of let-7b-5p, miR-184, and miR-22-3p showed a combined ROC AUC value of 92.4% (p<0.0001, FIG. 5). Furthermore, leave-one-out cross validation (LOOCV) analyses of the three predictor microRNAs (let-7b-5p. miR-184. miR-22-3p) showed an 80% match between the prediction and the outcome. Taken together, the above data indicate that these combined analytes (EV let-7b-5p, miR-184 levels combined with circulating miR-22-3p levels) robustly differentiate cancer patients from high-risk controls.

### Example 3. Analyses of miRNA-Related Treatment-Resistance Mechanisms

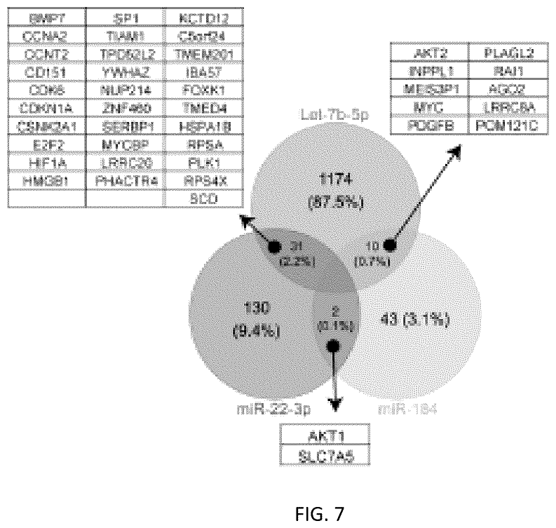

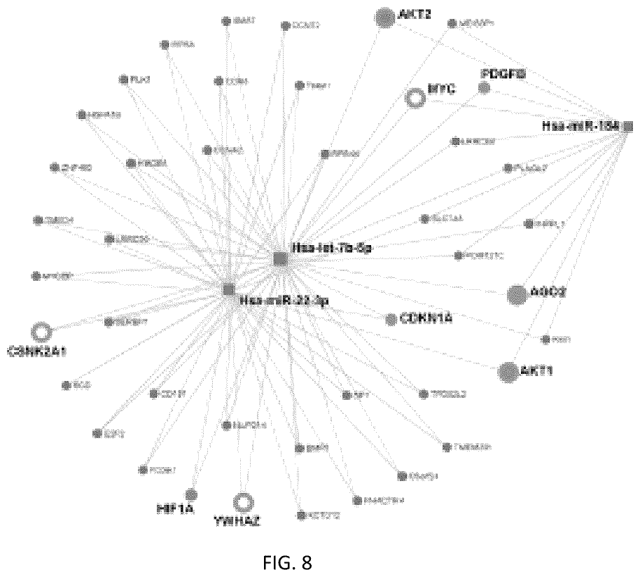

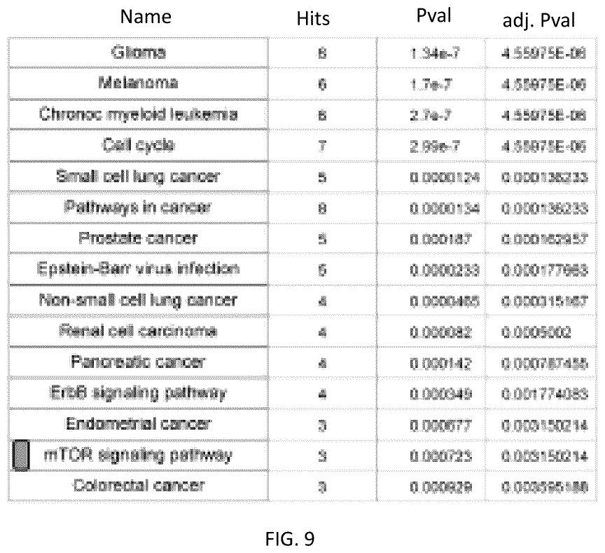

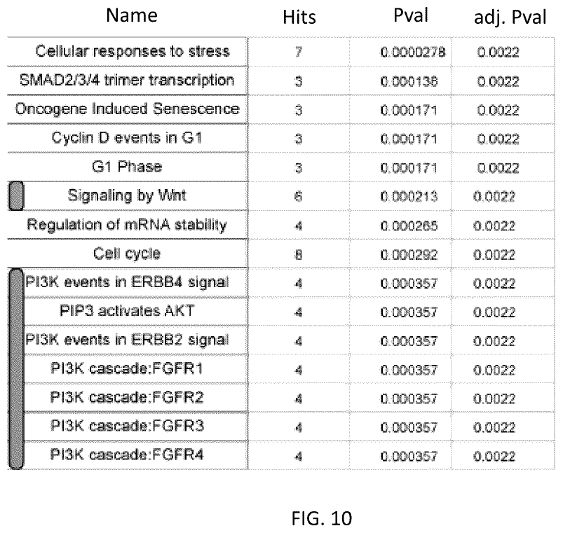

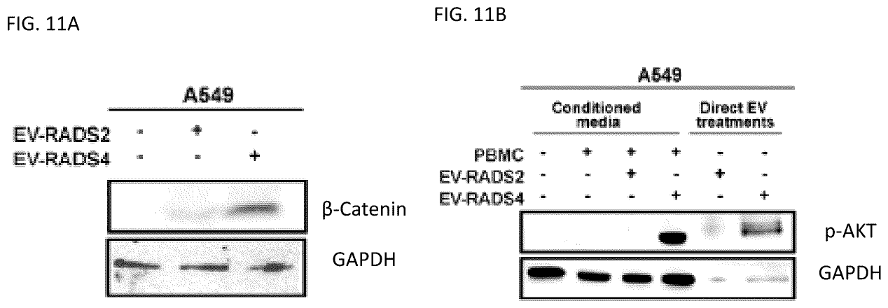

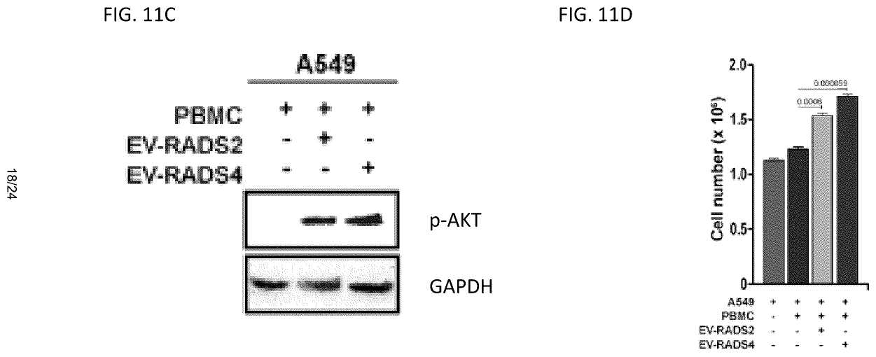

The possibility that these EV and circulating plasma miRNAs (let-7b-5p, miR-184, and miR-22-3p) mediate cell-cell communication events that support NSCLC disease progression was investigated. First, the miRNA target proteins analysis platform MIRNET was used to identify experimentally validated let-7b-5p, miR-184, and miR-22-3p target proteins. Proteins targeted by at least two of the three miRNAs from experimental data (MIRNET miR2gene) were prioritized, resulting the identification of 43 proteins (FIG. 7), which were subsequently interrogated in Gene Ontology analyses using Kyoto Encyclopedia of Genes and Genomes (KEGG) or Reactome classifications to derive signaling pathways. Cancer was the most highly enriched KEGG term (FIGS. 8 & 9), underscoring the robustness of the experimental pipeline and the relevance of these miRNAs to cancer disease. Interestingly, KEGG and Reactome signaling maps revealed that let-7b-5p, miR-184, and miR-22-3p converge on the activation of WNT and PI3K-AKT-mTOR signaling (FIGS. 9 & 10), suggesting that circulating and EV miRNAs cooperatively regulate WNT and PI3K-AKT-mTOR activity in NSCLC. Activation of WNT or PI3K-AKT-mTOR signaling in NSCLC tissues is associated with aggressive and therapy resistant disease. Considering that miR-184 and miR-22-3p are downregulated in cancer patients, this suggests that plasma from high-risk, yet cancer-free individuals contain EV and circulating miRNAs that suppress WNT and the AKT signaling axis and that these mechanisms are restrained in NSCLC patients. Indeed, treatment of NSCLC cells (A549) with cancer patients EV elevated WNT and AKT signaling levels compared to the effect of EV from screening controls, as determined by B-catenin and phospho-AKT protein levels, respectively (FIGS. 11A & 11B).

It is known that the uptake of EV by immune cells results in paracrine signaling loops that ultimately accelerate disease progression via complex mechanisms. To explore this possibility, supernatant transfer experiments were performed to determine whether cancer patients EV stimulate AKT/mTOR in A549 cells either directly or via immune cells. A549 cells were cultured in media conditioned by peripheral blood mononuclear cells (PBMC) left untreated or treated with EV either from controls or from cancer patients. Cancer patients EV/PBMC media dramatically stimulated phospho-AKT and phospho-mTOR levels in A549 cells, compared to controls (FIGS. 11B & 11C). EV from the high-risk controls also stimulated mTOR, possibly reflecting a NSCLC priming state (see discussion). Consistent with this AKT/mTOR stimulating potential, EV/PBMC conditioned media accelerated the growth of A549 cells (FIG. 11D). Taken together, the above data argue that plasma EV act directly or via immune cells to activate AKT in NSCLC.

Next, an analysis was conducted to determine whether let-7b-5p, miR-184, and miR-22-3p mediate the AKT activating effect of cancer EV and what implication this might have on NSCLC treatment outcomes. Activating mutations in Epidermal Growth Factor Receptor (EGFR) signaling represent one of the most known genetic alterations associated with NSCLC. Patients harboring sensitizing EGFR mutations (exon 19 deletion and L858R) respond favorably to first-and second-generation Tyrosine Kinase Inhibitors/TKI (gefitinib, erlotinib, afatinib, and dacomitinib). However, patients acquire TKI-desensitizing EGFR mutations (T790M) and become resistant to these TKIs. Osimertinib, a third generation TKI selectively targets EGFR T790M and generates significant clinical benefits in EGFR T790M patients. Unfortunately, all patients ultimately develop resistance to Osimertinib because they acquire an Osimertinib-desensitizing mutation (C797S) or activate complex compensatory signals to resist drug-induced cell death and to promote cancer cell proliferation. Understanding the nature of these signals and how they are activated have the potential to inform new treatment strategies for re-sensitizing patients to existing TKIs.

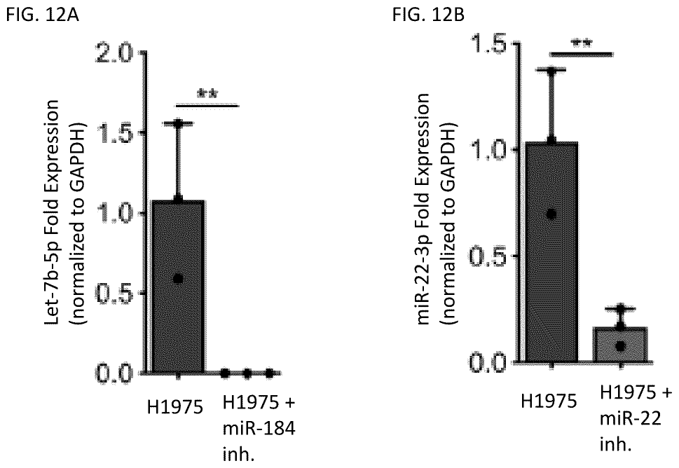

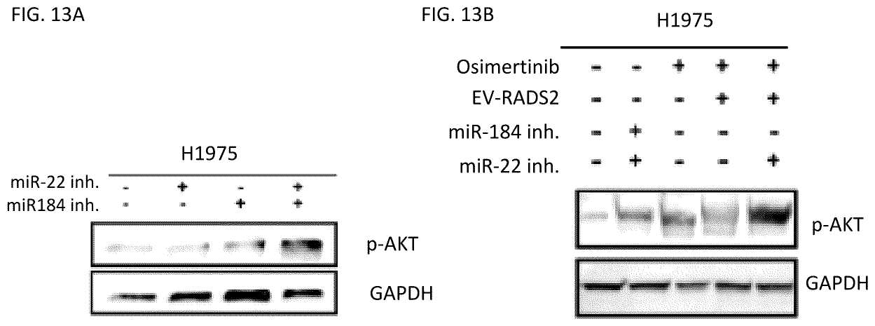

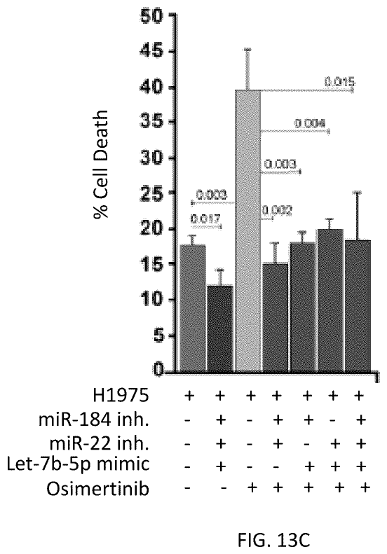

A role for let-7b-5p, miR-184, and miR-22-3p in NSCLC response to Osimertinib was investigated using H1975 NSCLC cells, which harbor L858R and T790M EGFR mutations. First, H1975 cells were transfected with miR-184 and miR-22-3p inhibitors, mimicking their reduction in cancer patient plasma, and the effect of miR-184/miR-22-3p inhibition on AKT activity assessed. The inhibitors reduced miR-184 and miR-22-3p levels in qPCR assays (FIGS. 12A & 12B, respectively) and cooperatively elevated pAKT levels in H1975 cells (FIGS. 13A & 13B). Reciprocal experiments demonstrated that Lung-RADS2 EV, which overexpress miR-184 (FIGS. 4C & 5B), inhibit Osimertinib-induced AKT activity in H1975 cells (FIG. 13B). Importantly, inhibition of miR-22-3p in this setting was sufficient to dramatically unleash AKT (FIG. 13B), further highlighting the cooperation between these two miRNAs in modulating AKT activity. Finally, the effect of miR-184/miR-22-3p inhibition on Osimertinib-induced cell death was investigated. Consistent with AKT stimulation, miR-184/miR-22-3p inhibition significantly suppressed Osimertinib-induced cell death (FIG. 13C). Similar results were observed when miR-184/miR-22-3p co-inhibited cells were treated in the presence of a let-7b-5p mimic (FIG. 13C).

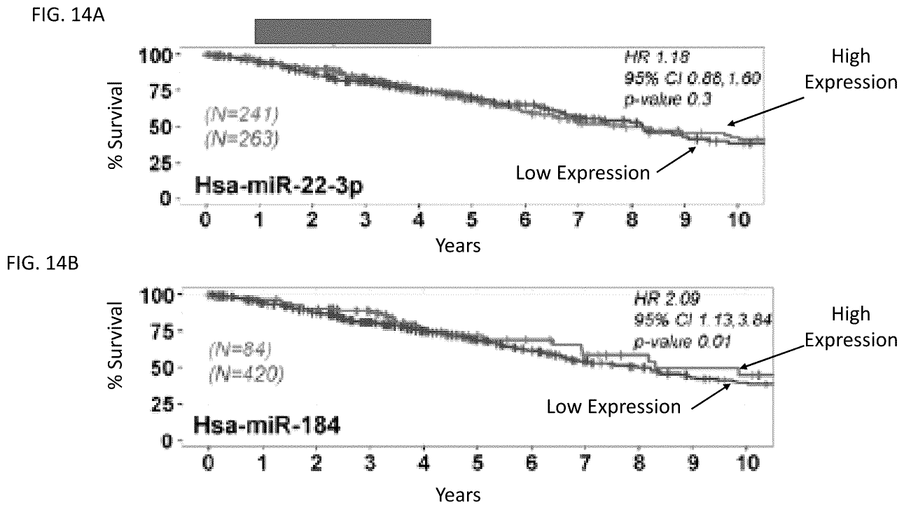

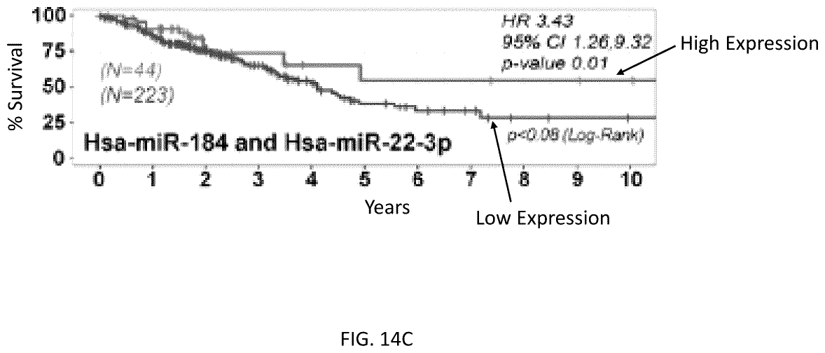

AKT activation is associated with NSCLC resistance to TKI, leading to reduced patient survival time. Thus, experiments were conducted to determine to what extent reduced miR-184/miR-22-3p tumor expression correlates with reduced patient survival using the cancer genome atlas (TCGA) LUAD patient tumor miRNA expression and survival data. Let-7 could not be combined with miR-22 or miR-184. Only a small number of patients (12) in the LUAD TCGA had combined expression data for Let-7 and miR-22/miR-184, making it difficult to reliably perform the analysis. No significant survival difference between patients whose tumors express low miR-22-3p (FIG. 14A) were detected. However, patients with low miR-184 tumor expression had a significantly shorter survival time compared to patients with higher miR-184 tumor expression (FIG. 14B, Hazard Ratio 2.09, 95% CI: 1.13, 3.84, p<0.018). Interestingly, patients with miR-184/miR-22-3p co-repressed tumors experienced even shorter survival time compared to patients with high miR-184/miR-22-3p tumor expression (FIG. 14C, Hazard Ratio 3.43, 95% CI: 1.26, 9.32, p<0.016). Note that the survivorship of miR-184/miR-22-3p tumor low patients is significantly lower than that of patients with tumor low miR-184 alone or miR-22-3p alone. This is consistent with AKT activation in miR-184/miR-22-3p co-inhibited NSCLC patients' plasma and treatment resistance.

These results demonstrate that EV (let-7b-5p, miR-184) and circulating (miR-22-3p) plasma miRNA likely modulate NSCLC response to Osimertinib, highlighting a novel mechanism of resistance and suggesting that these biomarkers may assist in the selection of patients that will likely benefit from Osimertinib/AKT blockade combination treatments.

### DISCUSSION OF RESULTS

The implementation of LDCT in NSCLC screening has reduced patient deaths by ˜20%. However, LDCT screening has a 23-50% false-positive rate, causing unnecessary exposure to radiation, costly and invasive follow-up studies for these mis-diagnosed patients. Thus, additional strategies are needed to improve NSCLC screening accuracy. The results disclosed herein identify of a set of miRNAs (let-7-5p, miR-184 from EV and miR-22-3p from circulating miRNAs) that distinguishes NSCLC patients from high-risk controls. Additionally, it was found that the EV markers CD9, CD63, but not CD81 or flotillin, are differentially expressed in NSCLC patients compared to high-risk controls.

Unique features of this study include the fact that reference and disease cohorts were controlled for risk profiles, reducing noise, and elevating the relevance of the identified biomarkers in NSCLC. Additionally, the present study integrates plasma and EV miRNAs to not only identify potential core molecular signatures for NSCLC risk management but to also highlight a plausible cooperation between these miRNAs in influencing patients' drug response. Combining multiple analytes of diverse biological origins (EV surface markers, EV, and plasma miRNAs) also maximizes robustness in diagnostics. Consistent with this, the disclosed biomarkers were able to differentiate confirmed NSCLC patients from LDCT false-positives, suggesting that addition of these non-invasive biomarkers to LDCT screening can improve diagnosis accuracy.

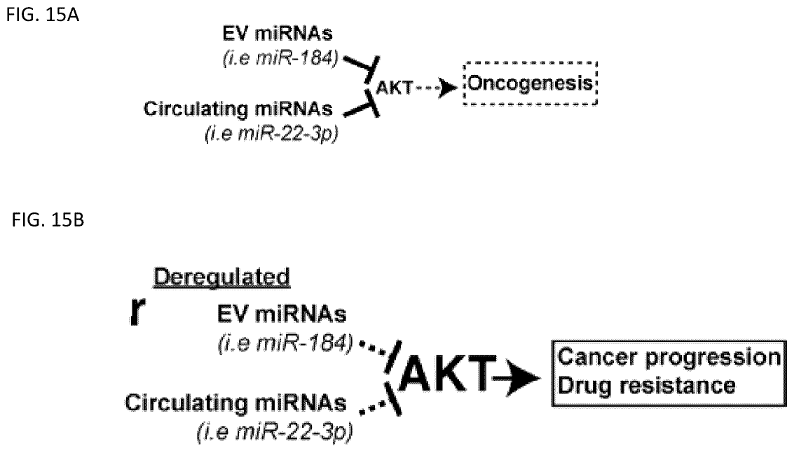

Confirmed NSCLC patients are stratified to diverse treatment options, including chemotherapy, immunotherapy, and targeted therapies based on histological and genetic mutations profiles obtained from tissue biopsies. Due to positional constraints, however, tissues biopsies often fail to capture the broader complexity of genetic driver mutations, leading to incomplete targeted therapy responses. The profiling of cancer-derived EV from patients' plasma may provide deeper insights into the overall cancer mutational landscape of the tumor and thus better guide treatment decisions in the future. Indeed, pathway analyses of let-7b-5p, miR-184, and miR-22-3p target proteins revealed that these miRNAs converge on therapy resistance signals, including AKT. Thus, circulating and EV miRNAs may functionally cooperate to modulate oncogenesis or patients' clinical outcomes (FIGS. 15A & 15B). The AKT-suppressing miRNAs miR-184/miR-22-3p function as tumor suppressors and delay oncogenesis (FIG. 15A). Cancers downregulate the expression of these miRNAs and/or reduce their systemic abundance via an unknown mechanism, leading to high baseline AKT activity and potentially resulting into accelerated cancer growth and drug resistance (FIG. 15B). Depending on context, EV and plasma miRNAs may act directly or via tumor-interacting immune components to modulate mTOR/AKT total protein levels or activity state (i.e., by downregulating upstream regulators of mTOR/AKT), thereby influencing tumor cell behavior. Congruent with these possible modes of action, targeting miR-184 or miR-22-3p moderately elevated total mTOR protein levels in A549 cells. While these treatments show no detectable effect on total AKT levels. They dramatically increased phospho-AKT levels in H1975 cells (FIGS. 13A & 13B).

Mimicking the expression profile of miR-184 (EV) and miR-22-3p (plasma) in NSCLC patients' blood using miRNA inhibitors cooperatively activated AKT and desensitized NSCLC cells (H1975) to Osimertinib. Thus, Let-7b-5p, miR-184, and miR-22-3p represent liquid biopsy biomarkers that may assist with the identification of patients that will likely benefit from Osimertinib/AKT blockade combination treatments. AKT inhibition re-sensitizes TKI-resistant NSCLC cells to erlotinib and gefitinib. A clinical trial evaluating the efficacy of combining Osimertinib with aspirin (an AKT inhibitor) in advanced NSCLC patients is pending (NCT04184921). Further, aberrant activation of AKT/mTOR and WNT/β-catenin signaling are associated with therapy resistance across different cancer types, suggesting broad translatability for these markers and their underlying mechanisms of action.

1. A method of identifying a test individual as having lung cancer, comprising:
i) comparing the level let-7b-5p miRNA in the control individual with the level of let-7b-5p miRNA in a test individual; and/or
ii) comparing the level miR-184 miRNA in the control individual with the level of miR-184 miRNA in a test individual; and/or,
iii) comparing the level miR-22-3p miRNA in the control individual with the level of miR-22-3p miRNA in a test individual;
wherein if the level of let-7b-5p miRNA in the test individual is significantly greater than the level of let-7b-5p miRNA in the control individual; and/or,
the level of miR-184 miRNA in the test individual is significantly less than the level of miR-184 miRNA in the control individual; and/or,
the level of miR-22-3p miRNA in the test individual is significantly less than the level of miR-22-3p miRNA in the control individual;
the individual is identified as having lung cancer.

2. The method of claim 1, wherein the let-7b-5p miRNA is EV-associated let-7b-5p miRNA.

3. The method of claim 1, wherein the individual is identified as having lung cancer if the level of let-7b-5p miRNA in the test individual is at least 0.5 logs greater than the level of let-7b-5p miRNA in the control individual.

4. The method of claim 1, wherein the miR-184 miRNA is EV-associated miR-184 miRNA.

5. The method of claim 1, wherein the individual is identified as having lung cancer if the level of miR-184 miRNA in the test individual is at least 20% lower than the level of miR-184 miRNA in the control individual.

6. The method of claim 1, wherein the miR-22-3p miRNA is circulating miR-22-3p miRNA.

7. The method of claim 1, wherein the individual is identified as having lung cancer if the level of miR-22-3p miRNA in the test individual is at least 20% lower than the level of miR-22-3p miRNA in the control individual.

8. The method of claim 1, wherein the control individual is a different individual than the test individual and is known to be free of lung cancer.

9. The method of claim 1, wherein the control individual is the same individual as the test individual, and the levels of let-7b-5p, miR-184, and miR-22-3p are determined at a time when the individual was known to be free of lung cancer.

10. The method of claim 1, wherein the levels of let-7b-5p, miR-184, and miR-22-3p are determined using a blood sample obtained from the test individual.

11. A method of decreasing the treatment-resistance of a lung cancer in a cancer patient, comprising modulating the expression of let-7b-5p miRNA, miR-184 miRNA, and/or miR-22-3p miRNA in the cancer patient.

12. The method of claim 11, wherein modulating the expression comprises a step selected from the group consisting of:
decreasing the level of let-7b-5p miRNA in the cancer patient; and/or
increasing the level of miR-184 miRNA in the cancer patient; and/or
increasing the level of miR-22-3p miRNA in the cancer patient.

13. The method of claim 12, wherein modulating the expression comprises administering miR-184 miRNA and/or miR-22-3p miRNA to the cancer patient.

14. The method of claim 12, wherein modulating the expression comprises administering a vector encoding miR-184 miRNA and/or miR-22-3p miRNA to the cancer patient.

15. A method of sensitizing lung cancer cells to treatment in a cancer patient, comprising modulating the expression of let-7b-5p miRNA, miR-184 miRNA, and/or miR-22-3p miRNA in the cancer patient.

16. The method of claim 15, wherein modulating the expression comprises a step selected from the group consisting of:
decreasing the level of let-7b-5p miRNA in the cancer patient; and/or
increasing the level of miR-184 miRNA in the cancer patient; and/or
increasing the level of miR-22-3p miRNA in the cancer patient.

17. The method of claim 15, wherein modulating the expression comprises administering miR-184 miRNA and/or miR-22-3p miRNA to the cancer patient.

18. The method of claim 15, wherein modulating the expression comprises administering a vector encoding miR-184 miRNA and/or miR-22-3p miRNA to the cancer patient,
